(19) United States
(12) Patent Application Publication
(10) Pub. No.: US 2014/0212988 A1
(43) Pub. Date: Jul. 31, 2014

(54) METHOD OF DETECTING AN ANALYTE USING COATED HOLLOW MICROSPHERES AND METHOD OF PRODUCING SAME

(71) Applicant: iFyber, LLC, Ithaca, NY (US)

(72) Inventors: Aaron D. Strickland, Freeville, NY (US); Robert Richard Diaz-Morales, Ithaca, NY (US); Yajaira Sierra-Sastre, Ithaca, NY (US)

(73) Assignee: iFyber, LLC, Ithaca, NY (US)

(21) Appl. No.: 13/917,107

(22) Filed: Jun. 13, 2013

Related U.S. Application Data
(63) Continuation of application No. PCT/US2011/065179, filed on Dec. 15, 2011.
(60) Provisional application No. 61/423,208, filed on Dec. 15, 2010.

(51) Int. Cl. G01N 33/543

(52) U.S. Cl. CPC G01N 33/54306
USPC 436/501

(57) ABSTRACT

Disclosed in this specification is a method for detecting an analyte using buoyant particles and chemical moieties to give buoyant particle composites that exhibit SERS and can be used for detecting the analytes in a liquid sample. A method is provided for detecting analytes of interest by contacting the analyte with a buoyant particle that comprises a first chemical moiety, such as a SERS-active component, allowing the analyte of interest to bind to the first chemical moiety. The resulting composite localizes in a discrete location of the liquid sample through a buoyant force. The composite is then detected by measuring the Raman scattered light in the discrete location of the liquid sample.

### CROSS-REFERENCE TO RELATED APPLICATIONS

This application is a continuation of international application no. US2011/065179 (filed Dec. 15, 2011) which claims priority to U.S. provisional patent application Ser. No. 61/423,208 (filed Dec. 15, 2010), which applications are incorporated herein by reference in their entirety.

### STATEMENT REGARDING FEDERALLY FUNDED RESEARCH OR DEVELOPMENT

This invention was made with Government support under contract W31P4Q-11-C-0223 awarded by Defense Advanced Research Projects Agency (DARPA). The Government has certain rights in the invention.

### FIELD OF THE INVENTION

This invention relates generally to methods and compositions for detecting analytes using buoyant particles and analyte specific coatings and methods of preparing buoyant particles used in detection systems.

### BACKGROUND

Rising costs associated with healthcare and environmental remediation and the general increase in the public's access to information has spawned a significant interest in cost effective tools for rapidly obtaining information regarding chemical and biological analytes relevant to human health and the environment, for example. Rather than obtain results from complicated and costly analyte detection systems within large, centralized laboratories, there is a commercial need for the development of decentralized testing platforms that can provide valuable, time-sensitive information in the field (e.g., emergency rooms, clinics, public venues, remote locations) often encountered by the military, first responders and medical professionals. Currently available platforms for detecting chemical and biological analytes are limited by factors associated with testing sensitivity, specificity, portability and cost.

As an example, the in vitro diagnostics (IVD) industry plays an integral role in healthcare and disease management; the current IVD market is estimated at $44B US. Testing growth in the IVD industry is driven by the increased demand by consumers to have rapid access to lab tests, especially in the case of time sensitive tests. This demand is fueled by an increasing number of people developing chronic illnesses such as diabetes cardiovascular disease, and by the globalization of infectious disease. Strategies are needed to meet the demands of the world IVD market that are time-sensitive, portable, cost-effective and provide the sensitivity and specificity available from centralized laboratory IVD testing.

The field of detecting chemical and biological analytes has used various types of analytical techniques coupled to a host of different materials for detection. For example, various integrated systems involving analytical end-point detection technologies and associated materials have been developed to facilitate analyte capture and detection. An example of a currently available integrated end-point detection system is the xMap® system (Luminex® XM), which uses mixtures of fluorescent molecules encapsulated in microsphere materials to indirectly detect biological analytes (e.g., antibodies, antigens, nucleotides, etc.) using fluorescence. In this system, analytes are captured on the surface of the microsphere particle using analyte-specific coatings, and subsequently localized to a specific location within the detection system using a microfluidic device, where the analyte bound microspheres are detected using fluorescence.

Microfluidic devices have a tendency to be difficult to operate with many sample matrices, and often require the use of aggressive sample preparation to remove unwanted debris that may clog the microfluidic channels. Alternatively, magnetic particles with analyte specific coatings can be used for analyte capture and localization using external magnetic fields. Unfortunately, magnetic capture techniques are typically ineffective in volumes greater than about 500 microliters due to the magnetic field's 1/r² dependence, which makes it difficult to effectively capture magnetic particles in larger volumes.

Fluorescence is a common end-point detection analytical technique, used widely in the IVD industry, environmental monitoring, and a host of other industries that regularly detect analytes of interest. Fluorescence is a sensitive technique and has been used to detect single molecules under specialized conditions. A typical fluorescence spectrum contains very little information and the broad bands associated with fluorescence limits its utility in multiplex detection and detection in complex sample matrices that may have competing fluorescence bands. Thus, analyte detection systems that utilize fluorescence typically require sample pretreatment to remove the analyte from any complicating matrix component.

The embodiments disclosed herein are directed at overcoming one or more of the limitations discussed above regarding methods for detecting analytes, and the production of materials to facility analyte capture, localization and detection.

### SUMMARY OF THE INVENTION

The invention pertains to combinations of buoyant particles, analytes and chemical moieties to give buoyant particle composites that exhibit SERS and can be used for detecting the analytes in a liquid sample. A method is provided for detecting analytes of interest by contacting the analyte with a buoyant particle that comprises a first chemical moiety, such as a SERS-active component, allowing the analyte of interest to bind to the first chemical moiety, allowing the resulting composite to localize in a discrete location of the liquid sample through a buoyant force, and detecting the analyte of interest by measuring the Raman scattered light in the discrete location of the liquid sample.

### BRIEF DESCRIPTION OF THE DRAWINGS

The present invention is disclosed with reference to the accompanying drawings, wherein:

FIG. 1 is a flow diagram of one exemplary method of detecting an analyte;

FIGS. 2A and 2B are illustrations of a buoyant particle composite;

FIGS. 2C and 2D are illustrations of a buoyant particle composite localizing in a discrete location;

FIGS. 2E and 2F are illustrations of a buoyant particle with a first chemical moiety binding to a Raman-active reporter component;

FIGS. 2G and 2H illustrate the buoyant particle composites localizing in a discrete location;

FIG. 3 shows the attachment of an analyte to a chemical moiety;

FIGS. 4A, 4B and 4C show the plasmon band of a buoyant particle composite;

FIG. 5 shows the effects of one exemplary detection method;

FIG. 6 depicts an exemplary sandwich assay;

FIG. 7 depicts an exemplary competitive assay;

FIG. 8 illustrates another exemplary competitive assay;

FIG. 9 shows another exemplary sandwich assay;

FIG. 10 depicts yet another exemplary sandwich assay;

FIG. 11 illustrates yet another exemplary sandwich assay;

FIGS. 12A-D show the another sandwich assay and its effects;

FIG. 13 depicts another exemplary competitive assay;

FIG. 14 shows yet another exemplary sandwich assay;

FIG. 15 is a flow diagram depicting a method of forming a buoyant particle composite;

FIGS. 16A, 16B and 16C are depictions of the formation of a buoyant particle;

FIGS. 17A, 17B and 17C are depictions of the formation of another buoyant particle; and

FIGS. 18, 19A, 19B, and 19C show formation of another buoyant particle; and

FIGS. 20A, 20B, 21A, and 21B show formation of another buoyant particle.

Corresponding reference characters indicate corresponding parts throughout the several views. The examples set out herein illustrate several embodiments of the invention but should not be construed as limiting the scope of the invention in any manner.

### DETAILED DESCRIPTION

A general system for detecting target analytes is provided, referred to herein as a lab-on-a-bubble (LoB) detection system, that comprises a buoyant particle, a first chemical moiety and an analyte bound to the buoyant particle by the chemical moiety. Detecting the target analyte is accomplished using a Raman spectrometer to measure the Raman scattered light in a discrete location within a sample.

When taking advantage of the phenomenon known as surface-enhanced Raman scattering (SERS), which occurs when species are localized next to gold or silver surfaces with nanoscale features, Raman detection provides an analytical technique that rivals fluorescence spectroscopy. Further, SERS offers broader multiplexing capabilities given the high information content and high spectral resolution of Raman spectra. Information content determines the number of multiplexed spectra that can be distinguished in an assay, and thus, the number of unique targets that can be detected.

The buoyant particle can be used to control the density of a buoyant particle composite that is used to detect target analytes. The buoyant particle can also be used to localize a buoyant particle composite to a discrete location within a sample to enhance the detection of target analytes. The SERS component is used to enhance the Raman signal of the Raman-active reporting component and can be a SERS-active conformal coating of nanoparticles on the surface of the buoyant particle. The SERS component can also be a plurality of individual SERS nanoparticle reporters comprised of a gold or silver core and at least one surface bound Raman-active component.

The Raman-active component can be any material having a detectable Raman spectral signature. The Raman detection component can be any device this is capable of sensing Raman scattered light and collecting a Raman spectrum. The information included in the below section of the specification outlines the various combinations of components of the present invention, methods of producing the combination of components, and methods of using the combination of components to detect target analytes using a LoB detection system.

### Methods of Detecting Target Analytes Using LoB Detection Systems

### General Description of a LoB Detection System

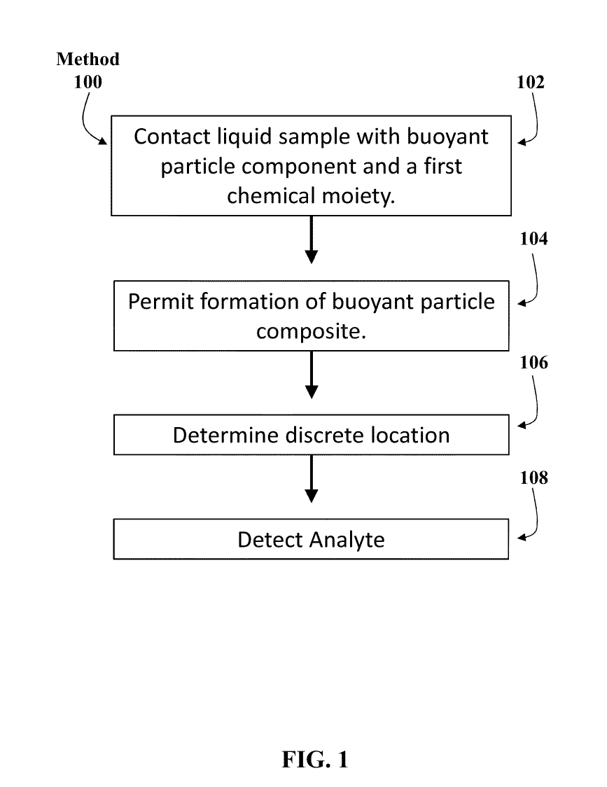

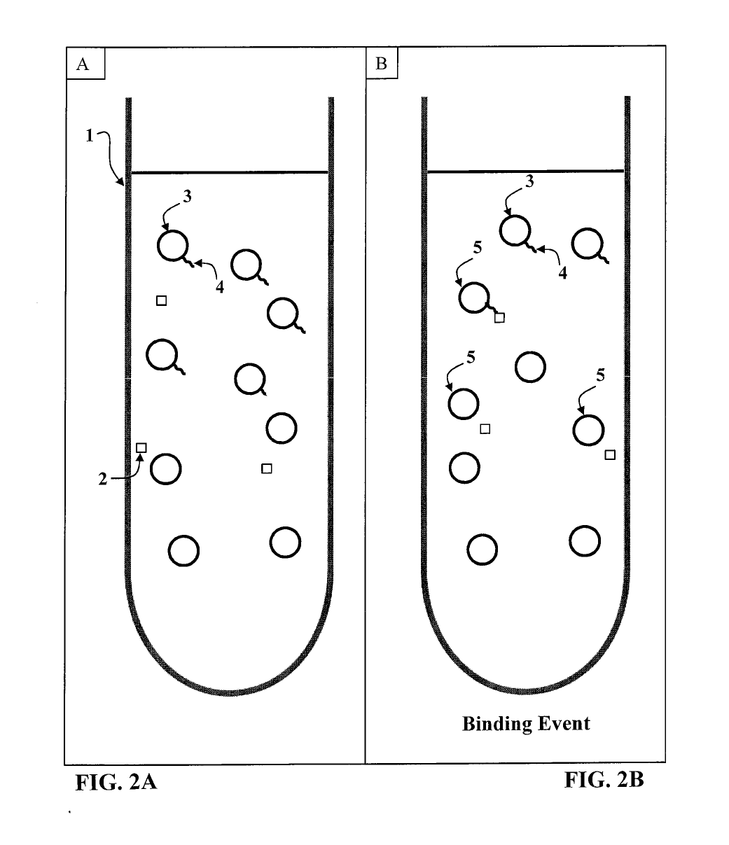

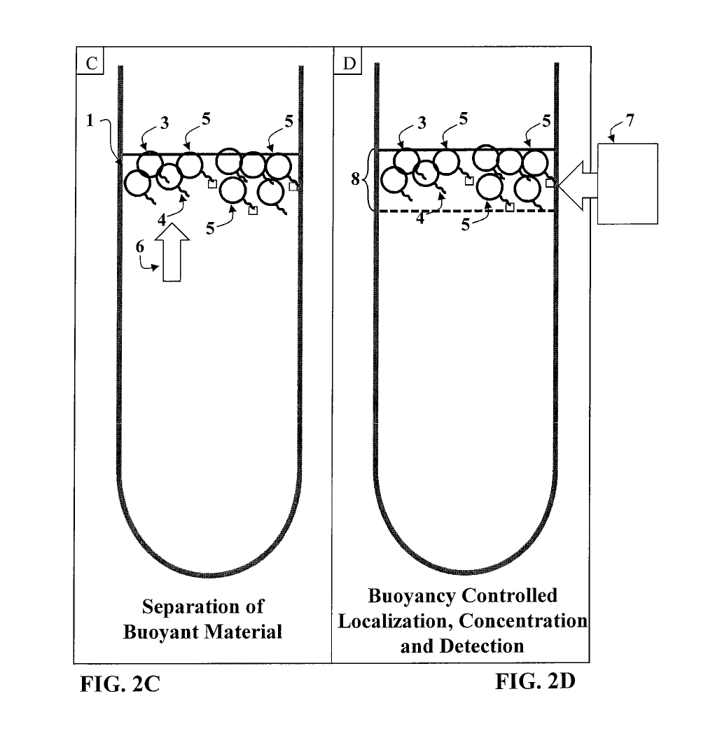

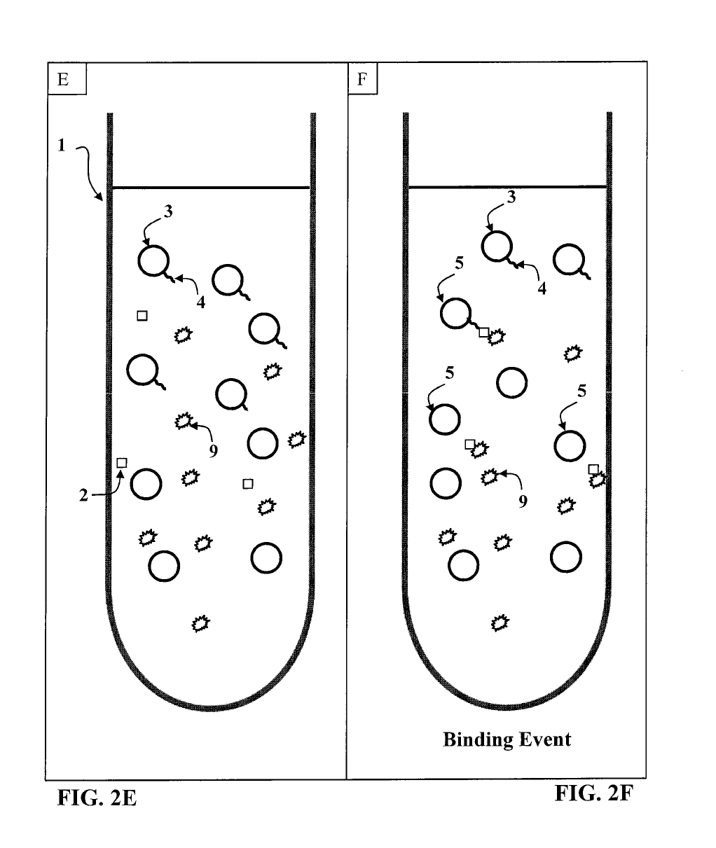

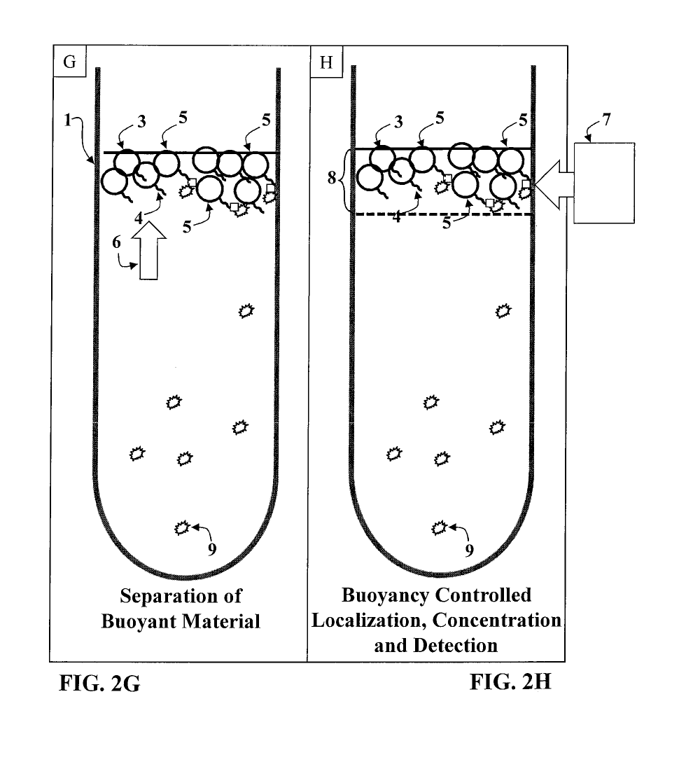

Referring to FIG. 1, a method 100 of detecting at least one analyte of interest in a sample is shown. The method 100 of FIG. 1 will be further described with reference to FIG. 2A-2H. The general architecture of a LoB system for detecting an analyte in a liquid sample is illustrated in FIGS. 2A-H. For simplicity, FIGS. 2A-H focus on two generic LoB systems for detecting an analyte 2. Those skilled in the art would recognize that the concepts presented in FIGS. 2A-H are merely representative, and that the LoB system can be adapted to many target analyte detection strategies using unique LoB systems. Detailed descriptions of other LoB systems are described in subsequent embodiments.

Referring to FIG. 1, in step 102, a liquid sample 1 is contacted with a buoyant particle 3. See FIG. 2A. The buoyant particle includes a first chemical moiety 4 chosen to selectively bind to an analyte of interest 2. Chemical moiety 4 is selected to bind to the surface of buoyant particle 3 and selectively bind to analyte 2. The chemical moiety 4 is disposed on the surface of the buoyant particle 3. In one embodiment, the chemical moiety 4 is disposed on the surface of buoyant particle 3 after the buoyant particle 3 and chemical moiety 4 have been mixed in liquid sample 1. The precise nature and composition of liquid sample 1, analyte 2, buoyant particle 3 and chemical moiety 4 are described in detail elsewhere in this specification.

Referring again to FIG. 1 and step 104 depicted therein, a buoyant particle composite 5 (see FIG. 2B) is permitted to form by allowing the analyte 2 to selectively bind to chemical moiety 4 and thereby form buoyant particle composite 5. See FIG. 2B. Buoyant particle composite 5 includes analyate 2, buoyant particle 3 and chemical moiety 4. The term “bind” refers to any chemical bond including hydrogen bonding, covalent bonding and ionic bonding. The term “selectively bind” refers to a chemical bond that forms selectively with a predetermined analyte. Examples of selective binding are provided elsewhere in this specification and include antigen-antibody binding as well as complementary hydrogen bonding.

Referring again to step 104, formation of the buoyant particle composite 5 is facilitated by mixing the ingredients throughout the liquid sample. The term “mixing” refer to any action including, but not limited to agitation, shaking, mechanical stirring, magnetic stirring, sample inversion, microfluidic mixing, sonication, or combinations thereof.

Referring again to FIG. 1 and step 106 depicted therein, buoyant particle composite 5 experiences a buoyant force 6 (see FIG. 2C) which causes it to become localized at a discrete location 8 (see FIG. 2D) in the liquid sample 1 based on its density. In step 106, this discrete location 8 is determined. In one embodiment, the discrete location 8 is at the top surface of the liquid sample 1. For example, the discrete location 8 may be determined to be at the top surface of the liquid sample 1.

In step 108 of FIG. 1 the discrete location 8 is interrogated for the presence of the bound analyte with a Raman spectrometer 7. In one embodiment, the composite particle is SERS active. In such an embodiment, interrogation of the discrete location 8 with Raman spectrometer 7 is particularly effective.

The general procedure outlined in FIG. 1 and FIGS. 2A-2D is utilized in a variety of methods to detect the analyte 2 using SERS. One such method is depicted in FIGS. 2E-2H.

Referring to FIGS. 2E to 2H, a liquid sample 1 is shown that includes a buoyant particle 3, a chemical moiety 4 and an analyte 2. FIG. 2E depicts the step of contacting the liquid sample 1 with this buoyant particle 3 and the chemical moiety 4 disposed thereon. The liquid sample 1 depicted in FIG. 2E also includes a Raman-active component 9. In one embodiment, the Raman-active component 9 is selected such that it tethers the Raman-active component 9 to the buoyant particle 3 to form buoyant particle composite 5 (step 104, FIG. 2F). In this fashion the presence of analyte 2 affects the number of Raman-active components 9 can be tethered to the buoyant particle 3 to form buoyant particle composite 5. In step 104, the buoyant composite 5 floats to a discrete location (see FIG. 2G). This is shown as discrete location 8 in FIG. 2H. Once the discrete location 8 has been determined (step 106) the analyte is detected (step 108) by interrogating discrete location 8 with spectrometer 7 (FIG. 2H).

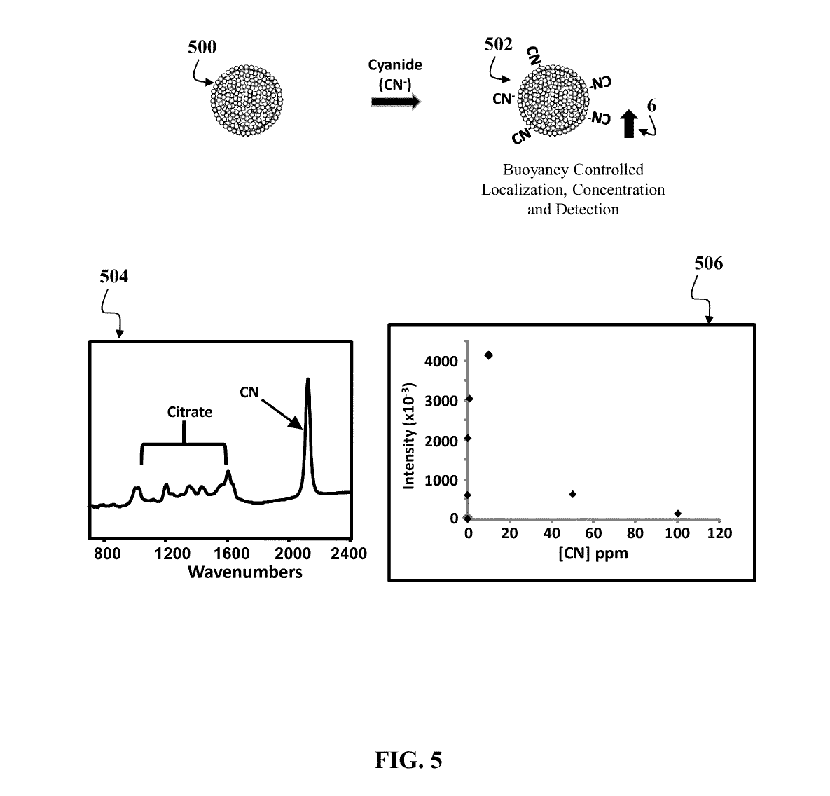

The target analyte 2 may be detected as a buoyant particle composite 5 comprised of a buoyant particle 3, a chemical moiety 4 and analyte 2. In one embodiment, the analyte is Raman-active and is detected by binding chemical moiety 4 to produce buoyant particle composite 5 having a Raman spectra signature. Methods that cover this aspect of the invention are described in more detail in FIG. 5. In another embodiment, the analyte may or may not be Raman-active, but is detected by binding to chemical moiety 4 (which is Raman-active) to produce buoyant particle composite 5 having a Raman spectral signature.

Buoyant particle 3 and buoyant particle composite 5 can be positively buoyant, such that it floats in a specified liquid within a specified period of time. For example, referring to water as the specified liquid, buoyant particles having a densities ranging between 0.1 and 0.9 grams per milliliter are considered positively buoyant particles and will float to the top of the vessel in short amount of time (e.g., within minutes). Buoyant particle 3 and buoyant particle composite 5 may also be neutrally buoyant. Buoyant particles having a densities ranging between 0.9 and 1.1 grams per milliliter are considered neutrally buoyant (again referring to water) and will remain suspended in the liquid over long periods of time (e.g., up to months).

Buoyant particle 3 can have a SERS-active coating comprised of a conformal coating of gold or silver particles. The SERS-active coating can affect the density of the underlying buoyant core material, whereby the addition of gold and or silver act to increase the density of the buoyant particle 3 relative to the buoyant core material.

Buoyant particle 3 can have a cross-sectional diameter that ranges between 0.1 micrometers and 500 micrometers, and has a density within the range of 0.1 and 1.5 g per mL. The buoyant particle 3 is comprised of a variety of materials including, but not limited to metal oxides (including silicon dioxide, aluminum oxide, titanium dioxide, or combinations of the same), organic polymers (including, but not limited to, polyethylene, polypropylene, sodium polystyrenesulfonic acid and latex) and metals (including silver, gold and combinations thereof) and gas-filled hollow particles.

In one embodiment, the buoyant particle 3 is a gas-filled hollow particle. By way of illustration buoyant particle 3 may be comprised of a silicon dioxide bubbles (e.g., 3M Corporation, Potters Industries, LLC Cospheric, LLC). Other examples of gas filled hollow particles are hollow glass microspheres having a surface coating of titanium oxide (e.g., Cospheric, LLC), hollow glass microspheres having a surface coating of silver (e.g., Cospheric, LLC), gas filled hollow particles comprised of alumina silicate cenospheres (e.g., Ceno Technologies), and silver coated alumina silicate cenospheres (e.g., Ceno Technologies).

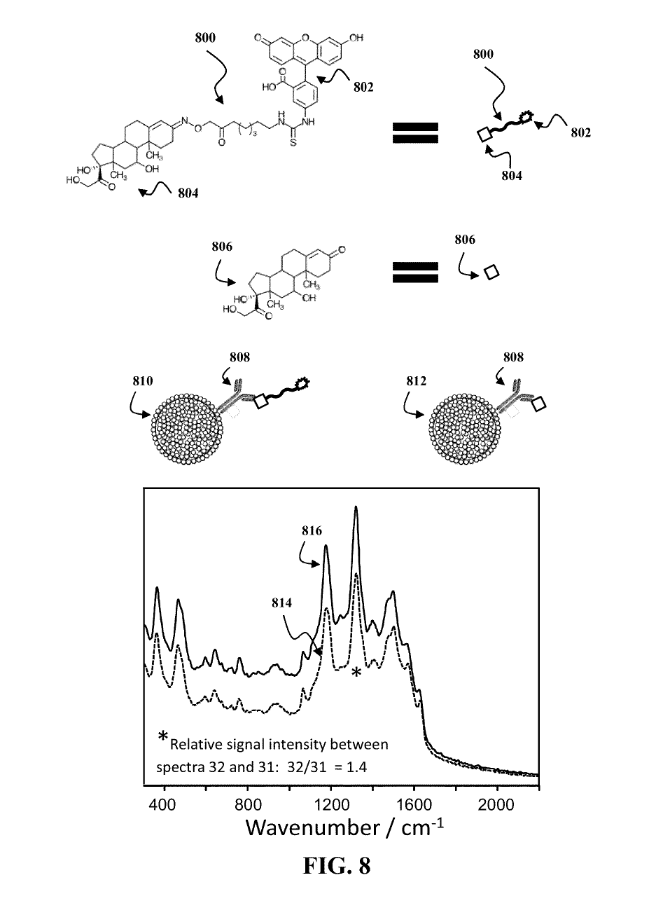

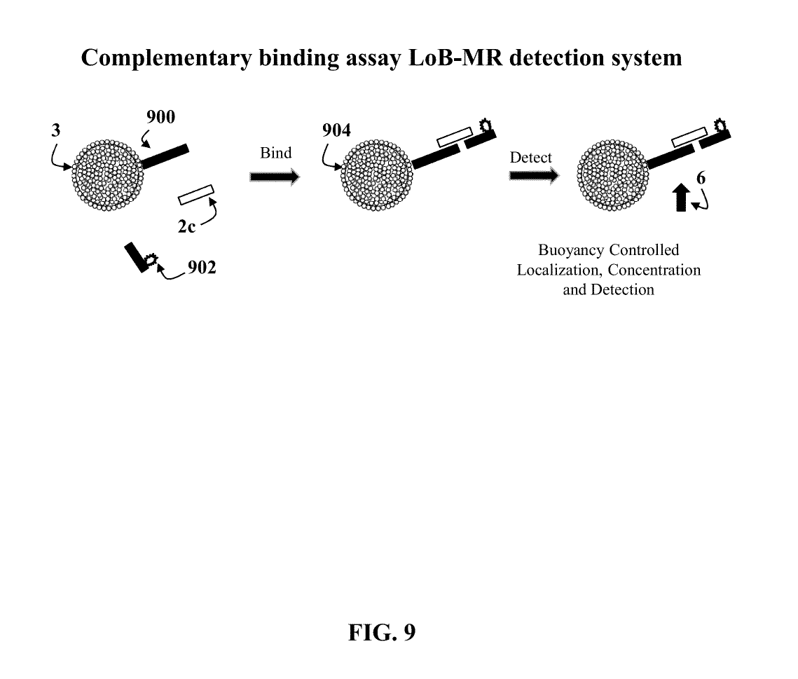

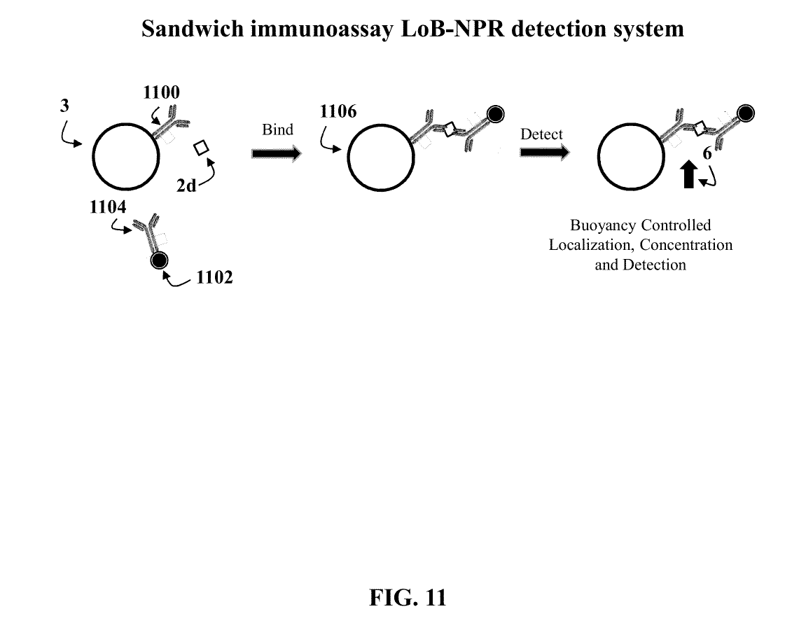

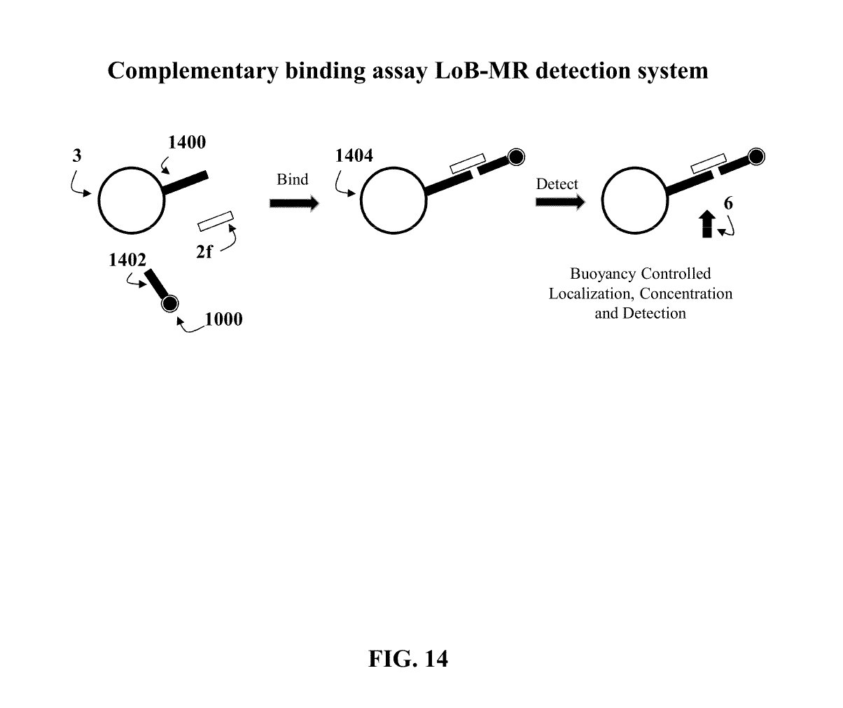

Analyte 2 may be a variety of substances including, but not limited to protein-based antigens (refer to FIG. 11), small organic molecule antigens (refer to FIG. 8, nucleotides (as described in FIGS. 9 and 14), molecular ions (e.g., as described in FIG. 5) cells (e.g., prokaryotic cells, eukaryotic cells, etc.), small molecule toxins, metal ions, and pharmaceutical compounds.

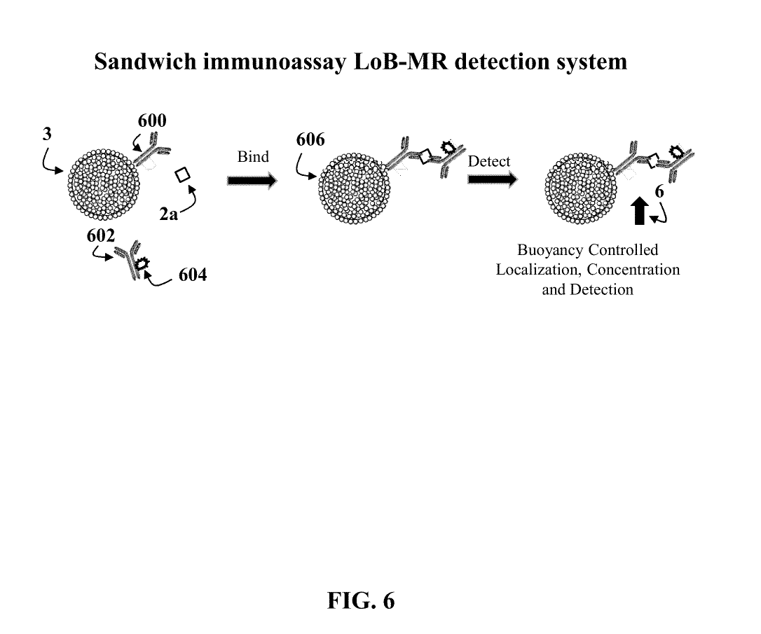

Chemical moiety 4 may be a variety of chemical groups that are capable of binding to the buoyant particle through hydrogen bonding, covalent bonding or ionic bonding. The chemical moiety 4 can be the surface of a SERS-active coating comprised of gold or silver particles (refer to FIG. 5), or can be a Raman-active component that is designed specifically to bind to an analyte. The chemical moiety can also be an antigen-binding antibody (refer to FIGS. 6-8 and 11-13), a nucleotide (refer to FIGS. 9 and 14), or an antibody-binding antigen.

Liquid sample 1 can comprise a variety of substances that are aqueous or non-aqueous mixtures having a density that is in the range of 0.1 and 2 g per mililiter. Liquid sample 1, can comprise a least one hydrocarbon solvent including, but not limited to, pentane, hexane, octane, crude oil, gasoline, ethanol, methanol, and combinations thereof. Liquid sample 1 can be a biological fluid or modified biological fluid including, but not limited to blood, plasma, serum, urine, sputum, saliva, and combinations thereof. Liquid sample 1 can comprise natural bodies of water that include, but not limited to oceans, lakes, rivers, streams, and seas. The sample may be potable water or waste water.

The present LoB detection system includes at least two general embodiments: LoB detection systems that utilize SERS-active buoyant particles and molecular reporters (herein referred to as a LoB-MR system), and LoB detection systems that utilize buoyant particles and SERS nanoparticle reporters (herein referred to as a LoB-NPR system). Each LoB detection system is explained below with specific reference to the various components that are utilized in the two LoB detection systems (e.g., buoyant particle component, a Raman-active component, a SERS component, as well as the types of target analytes that can be detected using the two LoB detection systems.

### Lab-on-a-Bubble Molecular Reporter (LoB-MR) System

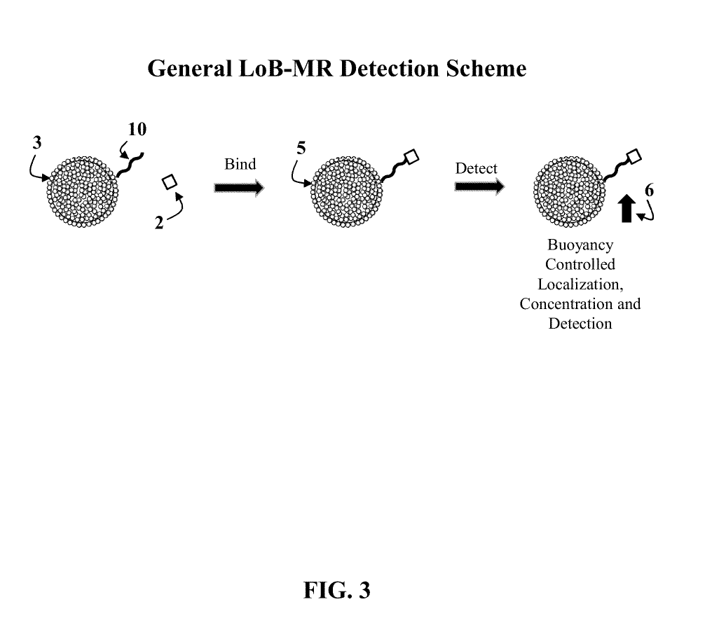

FIG. 3 illustrates a general schematic relating to steps involved in a LoB-MR detection system. In the LoB-MR detection system presented in FIG. 2, SERS-active buoyant particles 3 having a surface coating of chemical moiety 10 are combined with liquid sample that may or may not have a target analyte 2, and the components are mixed to allow for a binding event to occur between buoyant particle 3 and analyte 2. The resulting buoyant particle composite 5 is then allowed to localize and concentrate in the liquid sample via buoyant force 6 and subsequently detected according to the general method illustrated in FIG. 2.

In another embodiment, the buoyant particle 3 comprises a core material containing a plurality of surface binding agents capable of binding gold or silver particles, or combinations of the same, to give a conformal SERS-active coating. In one aspect of this embodiment, binding of the gold or silver particles requires binding agents with compatible chemistry for both the buoyant particle 3 and the gold or silver particles. Examples binding agents include, but are not limited to, organic polymers or a mixture of organic polymers that can bind both the core material and the gold or silver particles, organic molecules having a thiol, disulfide, or mercaptan group, an amine group, a pyridine group, a carboxylic acid group, a phosphate group, a trialkoxysilane silane group, a trichlorosilane group, or combinations thereof.

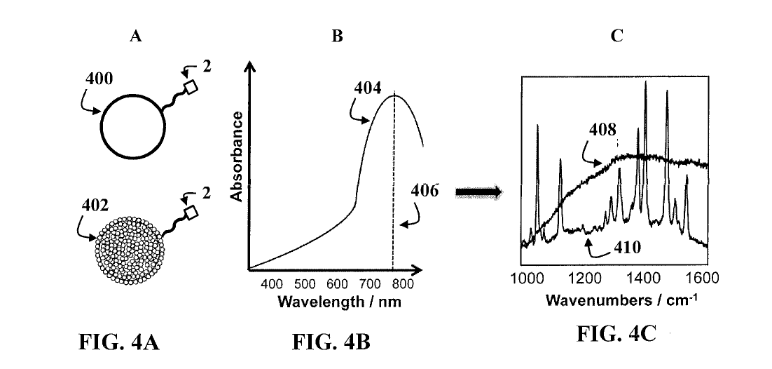

In another embodiment, the buoyant particle 3 is comprised of a SERS-active coating of gold and/or silver particles, or combinations of the same, that have a cross-sectional diameter within the range of 10 and 250 nanometers. Examples of gold or silver particles include, but are not limited to, spherical-shaped particles, rod-shaped particles and star-shaped particles. In one aspect of this embodiment, particle size, composition and interparticle distance can all be used, to give enhanced buoyant SERS-active composite particle, such that the average plasmon band of the buoyant SERS-active composite particle is in resonance with the excitation wavelength of the laser source of a Raman spectrometer used to detect a target analyte (refer to spectrometer 7 in FIG. 2D). This concept is illustrated in FIGS. 4A-C showing two different buoyant particle composites in FIG. 4A, wherein composite 400 is comprised of a buoyant particle devoid of any SERS-active coating and a bound target analyte 2 and composite 402 is a SERS-active buoyant particle bound to a target analyte 2. FIG. 4B further illustrates an absorption spectrum of a SERS-active buoyant particle composite 402 having a plasmon band 404 that is in resonance with the wavelength 406 of a laser source of a Raman spectrometer used to detect a target analyte 2. FIG. 4C further illustrates Raman spectra of composite 400 and composite 402, wherein spectrum 408 (corresponding to composite 400) is uninformative due to the lack of a SERS-active component in composite 408 and spectrum 410 (corresponding to composite 402) is rich in information and is a result of the SERS-active component of 402.

In another embodiment, the chemical moiety 10 of the SERS-active buoyant particle 3 illustrated in FIG. 3 is chosen such that it facilitates the binding of a target analyte 2. In one aspect of this embodiment, the SERS-active coating can function as the chemical moiety 10 if it has inherent binding affinity for the target analyte 2. In these cases, the target analyte 2 is the molecular reporter component of the LoB-MR system and can be detected if said target analyte 2 has a unique Raman signature. Examples of target analytes that can inherently bind to gold or silver particles include, but are not limited to, molecular anions, thiol-containing compounds, amine containing compounds, compounds having a lone pair of electrons, compounds having heteroaromatic rings, and combinations thereof. Specific examples include, but are not limited to cyanide, melamine, pyridines, mercaptopyridines, and aminothiophenol.

An example of such an embodiment is provided in FIG. 5, wherein the analyte 2 is a cyanide ion, the buoyant particle 3 is a SERS-active buoyant particle, and the chemical moiety 10 is a SERS-active coating of gold nanoparticles. In the embodiment depicted, the chemical moiety 10 (which doubles as a SERS-active coating) is disposed on the surface of buoyant particle 3 to form an intermediate particle 500 prior to introduction to the liquid sample 1. After introduction to the liquid sample, cyanide ions selectively bind to the chemical moiety 10 to produce buoyant particle composite 502. The Raman signature of analyte 2 is then detected by interrogating discrete location 8 with a Raman spectrometer according to the general method illustrated in FIG. 2. By way of illustration, Raman spectrum 504 is obtained from buoyant particle composite 502, which indicates a Raman spectral band for cyanide, and Raman spectral bands for citrate, which is also present in the sample. Plot 506 represents a titration curve that indicates the Raman spectral intensity of the cyanide Raman spectral band collected from various liquid samples containing a constant amount of SERS-active buoyant particles and varying cyanide concentrations (shown as ppm, or parts-per-million). As shown, the Raman spectral intensity for cyanide increases with increasing cyanide concentration. The decrease in Raman spectral intensity beyond a concentration of 20 ppm is gold dissolution from the buoyant particle surface, and thus, loss of SERS activity.

In another aspect of the previous embodiment, the chemical moiety 10 illustrated in FIG. 3 can be a synthetic molecular sensor that is designed to undergo specific chemical reaction with a specified target analyte or class of target analyte. In this aspect of the invention, the synthetic molecular sensor has a known Raman spectral signature, and upon reacting with a target analyte, a different Raman spectral signature results. In this aspect of the invention, the synthetic molecular sensor functions as the molecular reporter in this specific LoB-MR detection system. Exemplary methods for creating synthetic molecular sensors for detecting target analytes using Raman spectroscopy and SERS-active substrates are well known and can be adapted to the LoB-MR detection system disclosed herein. An example of such a method is described by Sulk et. al. (Journal of Raman Spectroscopy 1999, 30, (9), 853-859) for the detection of bilirubin and salicylate. The novel aspect of the present invention is the method for detecting target analytes using synthetic molecular sensors following the steps presented in general method illustrated in FIG. 2.

In another aspect, the chemical moiety 10 illustrated in FIG. 3 can comprise a functional antibody that has affinity for a analyte 2. For this aspect of the invention, the analyte 2 is referred to as an antigen and the LoB-MR detection system works on the basis of detecting the antigen indirectly in either a sandwich immunoassay, or a competitive immunoassay.

FIG. 6 illustrates a LoB-MR detection system using a sandwich immunoassay. A SERS-active buoyant particle 3 having a first chemical moiety 600 comprised of a first antibody is combined with a second chemical moiety 602 comprised of a second antibody and a liquid sample that may or may not have an analyte 2a. In the embodiment depicted in FIG. 6, buoyant particle 3 includes a SERS-active coating 608. SERS-active coating 608 may be gold and/or silver particles as described elsewhere in this specification. Second chemical moiety 602 may be the same or different than the first chemical moiety 600, but has affinity for the analyte 2a and also has Raman-active component 604 that exhibits a known Raman spectral signature, wherein component 604 functions as the molecular reporter in this specific LoB-MR detection system. The buoyant particle 3 (with first chemical moiety 600) is mixed with the liquid sample that includes second chemical moiety 602 (with Raman-active component 604) and (potentially) analyte 2a to allow for a binding event to occur between first chemical moiety 600, analyte 2a and second chemical moiety 602. This binding event tethers the Raman-active component 604 to the buoyant particle 3. The resulting buoyant particle composite 606 is then allowed to localize in the liquid sample via buoyant force 6. Composite 606 is subsequently detected according to the general method illustrated in FIG. 2.

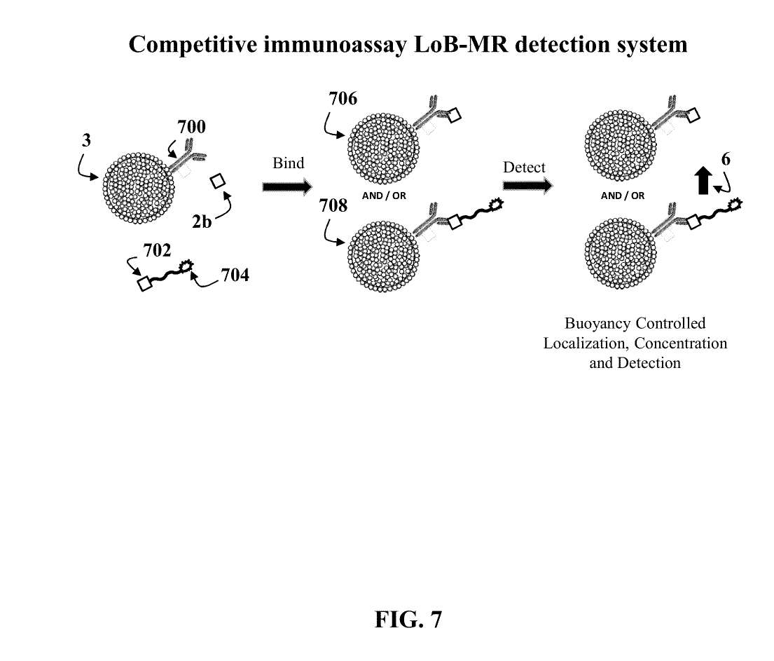

FIG. 7 illustrates a LoB-MR detection system that makes use of a competitive immunoassay, wherein a SERS-active buoyant particle 3 having a chemical moiety 700 comprised of an antibody is combined with a competing antigen 702 and a liquid sample that may or may not have an analyte 2b (an antigen). Competing antigen 702 has a known binding affinity to chemical moiety 700 and also has a Raman-active component 704 that exhibits a known Raman spectral signature and functions as the molecular reporter in this specific LoB-MR detection system. The components are mixed to allow for a binding event to occur between buoyant particle 3 and analyte 2b to give buoyant particle composite 706 or between buoyant particle 3 and competing antigen 702 to give buoyant particle composite 708. The buoyant particle composites 706 and 708 are then allowed to localize in the liquid sample via buoyant force 6 and subsequently detected by way of monitoring the Raman spectral signature of component 704 according to the general method illustrated in FIG. 1 and FIG. 2. In this particular LoB-MR assay, analyte 2b is detected indirectly by determining the ratio of buoyant particle composites 706 to 708 using the intensity of the Raman spectral signature of Raman-active component 704. As an example, in the presence of analyte 2b, the signal intensity obtained for Raman-active component 704 will be lower due to the fact analyte 2b competes with competing antigen 702 in binding chemical moiety 700 on buoyant particle 3.

By way of illustration, in one embodiment of the invention, a LoB-MR detection system is disclosed for cortisol detection using a competitive immunoassay (e.g., FIG. 8). In this aspect of the invention, a synthetic cortisol derivative which functions as a competing antigen 800 containing a fluorescein functional group 802 and a cortisol functional group 804 is combined in a liquid sample with a SERS-active buoyant particle having a first chemical moiety 808 (an antibody against cortisol) that is bound to the particle surface. First chemical moiety 808 binds competing antigen 800 to give buoyant particle composite 810 and can also bind free cortisol 806 to give buoyant particle composite 812. The presence of cortisol 806 in a liquid sample changes the relative amounts of composites 810 and 812, and this ratio can be quantified by monitoring the difference in spectral intensity of fluorescein functional group 802 after the various components are mixed and allowed to localize in the liquid sample according to the general method illustrated in FIG. 1 and FIG. 2. FIG. 8 shows spectra obtained for this assay in the presence 814 or absence 816 of free cortisol 806. In this way, free cortisol 806 can be detected in a liquid sample.

In another aspect of the previous embodiment, the chemical moiety 10 illustrated in FIG. 3 can comprise a functional deoxyribonucleic acid (DNA) molecule or a ribonucleic acid (RNA) molecule that has affinity for a analyte. For this aspect of the invention, the analyte is either a DNA molecule or a RNA molecule and the LoB-MR detection system works on the basis of detecting the analyte indirectly in a complementary binding assay. By way of illustration, FIG. 9 shows a LoB-MR detection system involving a SERS-active buoyant particle 3 having a chemical moiety 900 comprised of DNA or RNA, a analyte 2c comprised of DNA or RNA, and Raman-active component 902, which is comprised of DNA or RNA and has a known Raman spectral signature. In this aspect of the invention, chemical moiety 900 and Raman-active component 902 bind to complementary regions of analyte 2c, and the Raman-active component 902 functions as the molecular reporter in this specific LoB-MR detection system. The components are mixed to allow for a binding event to occur between SERS-active buoyant particle 2, analyte 2c and Raman-active component 902, and the resulting buoyant particle composite 904 is then allowed to localize and concentrate in the liquid sample via buoyant force 6 and subsequently detected by way of monitoring the Raman spectral signature of Raman-active component 902 according to the general method illustrated in FIG. 2.

### Lab-on-a-Bubble Nanoparticle Reporter (LoB-NPR) System

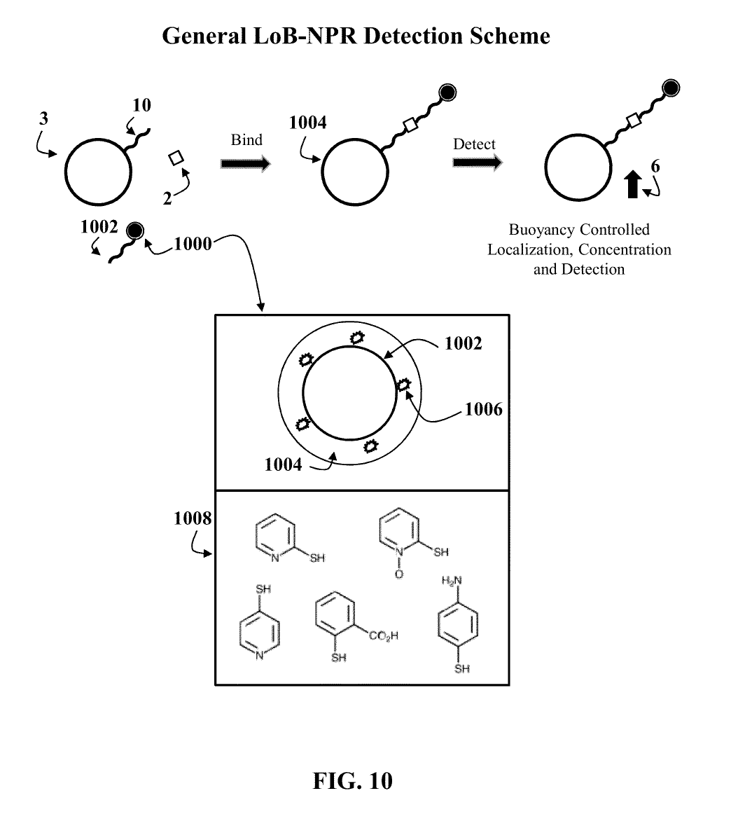

FIG. 10 illustrates a general schematic relating to steps involved in a LoB-NPR detection system. The LoB-NPR detection system presented in FIG. 10 is comprised of a buoyant particle 3 having a surface coating of chemical moiety 10. Particle 3 is combined with a SERS nanoparticle reporter 1000 having a second chemical moiety 1002 and a known Raman spectral signature, and also combined with a liquid sample that may or may not contain a analyte 2. The ingredients are mixed to allow for a binding event to occur between particle 3, analyte 2, and component 1000. The resulting buoyant particle composite 1004 is then allowed to localize in the liquid sample via buoyant force 6 and subsequently detected according to the general method illustrated in FIG. 1 and FIG. 2.

In one embodiment, the SERS nanoparticle reporter 1000 is comprised of a SERS-active gold or silver particle core 1002 and a protective shell of silicon dioxide 1004, wherein the SERS nanoparticle reporter also has a known Raman spectral signature. Examples of SERS nanoparticle reporter 1000 include, but are not limited to, those described by Doering and Nie (Analytical Chemistry 2003, 75, 6171-6176) and Mulvaney et. al. (Langmuir, 2003, 19, 4784-4790). Examples of gold or silver particle cores include, but are not limited to, spherical-shaped particles, rod-shaped particles and star-shaped particles with a cross-sectional diameter within the range of 10 and 250 nanometers. The nanoparticle reporter components can comprise a coating of Raman-active compound 1006 on the surface of the gold or silver core 1002, which is also encapsulated in a protective shell of silicon dioxide 1004, for example, and provides the SERS nanoparticle reporters 1000 with a known Raman spectral signature. Examples of Raman-active compounds 1006 are shown in FIG. 10 as 1008.

In another embodiment, the chemical moiety 10 has compatible chemistry for binding both buoyant particle 3 and an analyte 2. In an aspect of this embodiment, the chemical moiety 10 illustrated in FIG. 10 can comprise a functional antibody that has affinity for an analyte. For this aspect of the invention, the analyte is referred to as an antigen and the LoB-NPR detection system works on the basis of detecting the antigen indirectly in either a sandwich immunoassay, or a competitive immunoassay.

FIG. 11 illustrates a LoB-NPR detection system using a sandwich immunoassay. In one such sandwich immunoassay, a first chemical moiety 1100 and a second chemical moiety 1104 are provided which are peptides. These peptides are chosen to selectively bind to analyte 2d. In one embodiment, analyte 2d is an antigen and first and second chemical moieties 1100 and 1104 are antibodies chosen to selectively bind to analyte 2d. In another embodiment, analyte 2d is an antibody and first and second chemical moieties 1100 and 1104 are antigens chosen to selectively bind to analyte 2d. For example, a buoyant particle 3 having a chemical moiety 1100 comprising a first antibody is combined with a first nanoparticle reporter 1102 having a second chemical moiety 1104 comprising a second antibody and a liquid sample that may or may not have an analyte 2d. Second chemical moiety 1104 may be the same or different than the first chemical moiety 1100, but has known affinity for the analyte 2d. The ingredients are mixed to allow for a binding event to occur between first chemical moiety 1100, analyte 2d and second chemical moiety 1104. The resulting buoyant composite 1106 is allowed to localize in the liquid sample via buoyant force 6 and is subsequently detected according to the general method illustrated in FIG. 1 and FIG. 2.

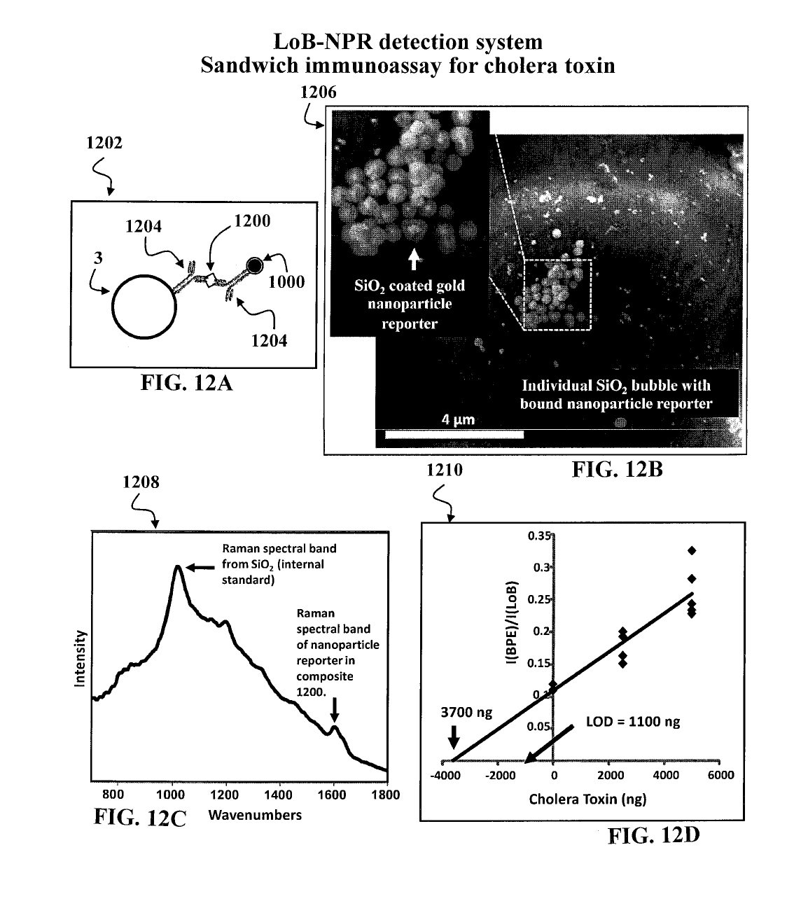

By way of illustration, in one embodiment of the invention, a LoB-NPR detection system is disclosed for cholera toxin detection using a sandwich immunoassay as generally outlined in FIG. 11. In this aspect of the invention, first chemical moiety 1100 and second chemical moiety 1104 are both an antibody specific for binding a cholera toxin antigen (i.e., anti-cholera antibody). Thus, as illustrated in FIG. 12A, in the presence of cholera toxin antigen 1200, a buoyant particle composite 1202 is formed that is comprised of a buoyant particle 3 having an anti-cholera antibody coating 1204, cholera toxin 1200, and a SERS nanoparticle reporter 1000 having an anti-cholera antibody coating 1204. Cholera toxin is then detected indirectly through buoyant particle composite 1202 after the various sandwich immunoassay ingredients are mixed, allowed to localize in the liquid sample according to the general method illustration in FIG. 1 and FIG. 2.

By way of further illustration, FIG. 12B shows scanning electron microscopy images 1206 of buoyant particle composite 1202 formed in a liquid sample containing cholera toxin. Also shown in FIG. 12C is a representative spectrum 1208 of a liquid sample containing buoyant particle composite 1202, and titration curve 1210 (FIG. 12D) used to quantify cholera toxin using a LoB-NPR detection system. Details of this specific aspect of the invention are covered in Example 7 disclosed herein.

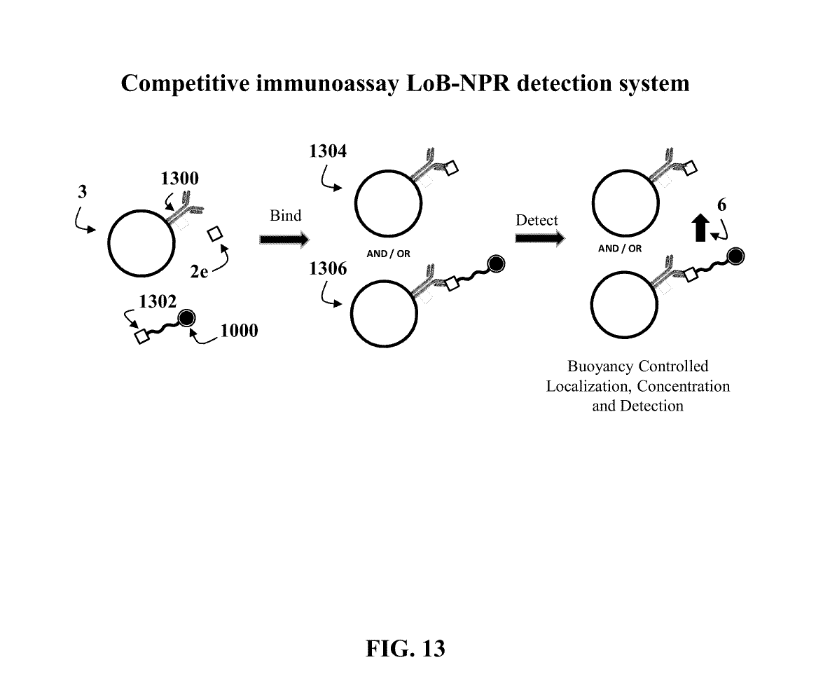

FIG. 13 illustrates a LoB-NPR detection system that makes use of a competitive immunoassay, wherein a buoyant particle 3 having a chemical moiety comprised of an antibody 1300 and a SERS nanoparticle reporter 1000 having a chemical moiety comprised of competing antigen 1302 are combined with a liquid sample that may or may not have an analyte 2e. Competing antigen 1302 has a known binding affinity to antibody 1300. The components are mixed to allow for a binding event to occur between buoyant particle 3 and analyte 2e to give buoyant particle composite 1304 or between buoyant particle 3 and competing antigen 1302 to give buoyant particle composite 1306. The buoyant particle composites 1304 and 1306 are then allowed to localize in the liquid sample via buoyant force 6 and subsequently detected by way of monitoring the Raman spectral signature of 1000 according to the general method illustrated in FIG. 1 and FIG. 2. In this particular LoB-NPR detection system, analyte 2e is detected indirectly by determining the ratio of buoyant particle composites 1304 to 1306 using the intensity of the Raman spectral signature of SERS nanoparticle reporter 1000. As an example, in the presence of target analyte 2e, the signal intensity from SERS nanoparticle reporter 1000 will be lower due to the fact analyte 2e competes with competing antigen 1302 in binding antibody 1300 on buoyant particle 3.

In another embodiment, the chemical moiety 10 illustrated in FIG. 10 can comprise a functional deoxyribonucleic acid (DNA) molecule or a ribonucleic acid (RNA) molecule that has affinity for an analyte. For this aspect of the invention, the analyte is either a DNA molecule or a RNA molecule and the LoB-NPR detection system works on the basis of detecting the analyte indirectly in a complementary binding assay. By way of illustration, FIG. 14 shows a LoB-NPR detection system in which a buoyant particle 3 having a chemical moiety 1400 comprised of DNA or RNA is combined with a SERS nanoparticle reporter 1000 and a liquid sample that may or may not have an analyte 2f. In this aspect of the invention, the SERS nanoparticle reporter 1000 has a surface coating 1402 comprised of DNA or RNA. Surface coating 1402 and chemical moiety 1400 bind to complementary regions of analyte 2f. The components are mixed to allow for a binding event to occur between chemical moiety 1400, analyte 2f and surface coating 1402, and the resulting buoyant composite material 1404 is then allowed to localize in the liquid sample via buoyant force 6 and subsequently detected by way of monitoring the Raman spectral signature of SERS nanoparticle reporter 100 according to the general method illustrated in FIG. 2.

In another embodiment, the general methods illustrated in FIG. 1 and FIG. 2 can be used to simultaneously detect one or more different analytes 2 simultaneously by contacting the liquid sample 1 with a plurality of different buoyant particles 3 having a plurality of different chemical moieties 4 that each specifically bind to a given analyte and/or specifically bind to a given Raman-active component 9 to give a plurality of different buoyant particle composites 5. The one of more analytes are detected based on the known Raman spectral signatures of the different buoyant particle composites.

### Method of Preparing SERS-Active Buoyant Particles

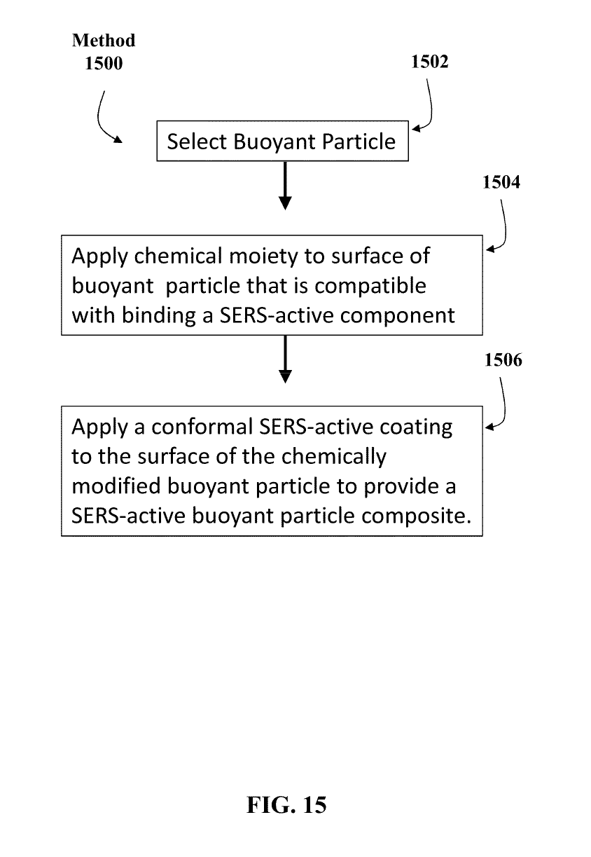

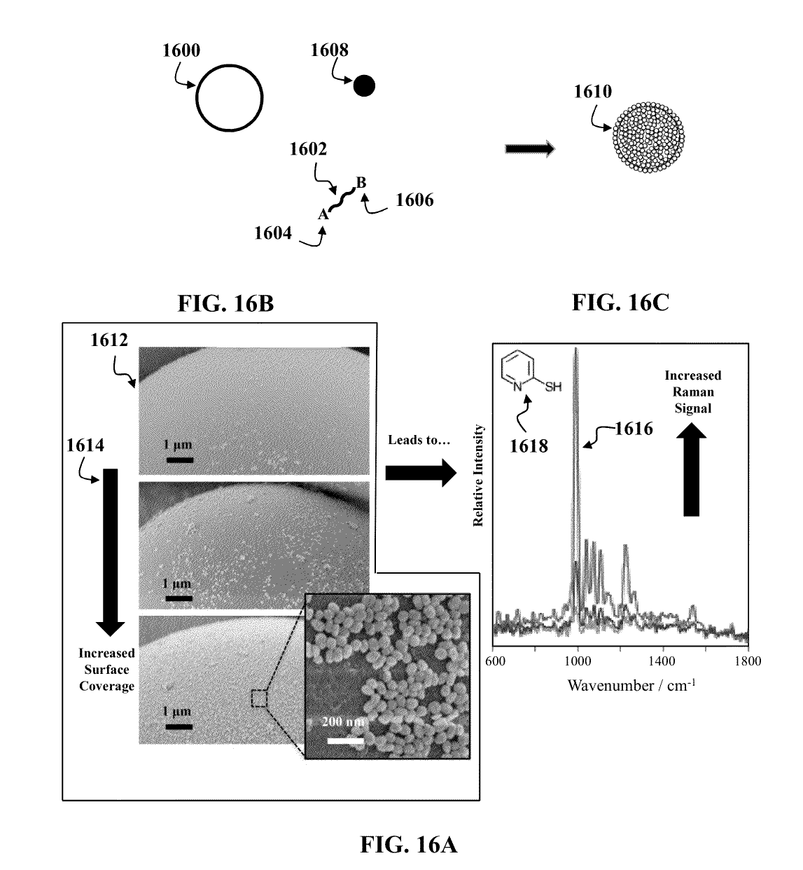

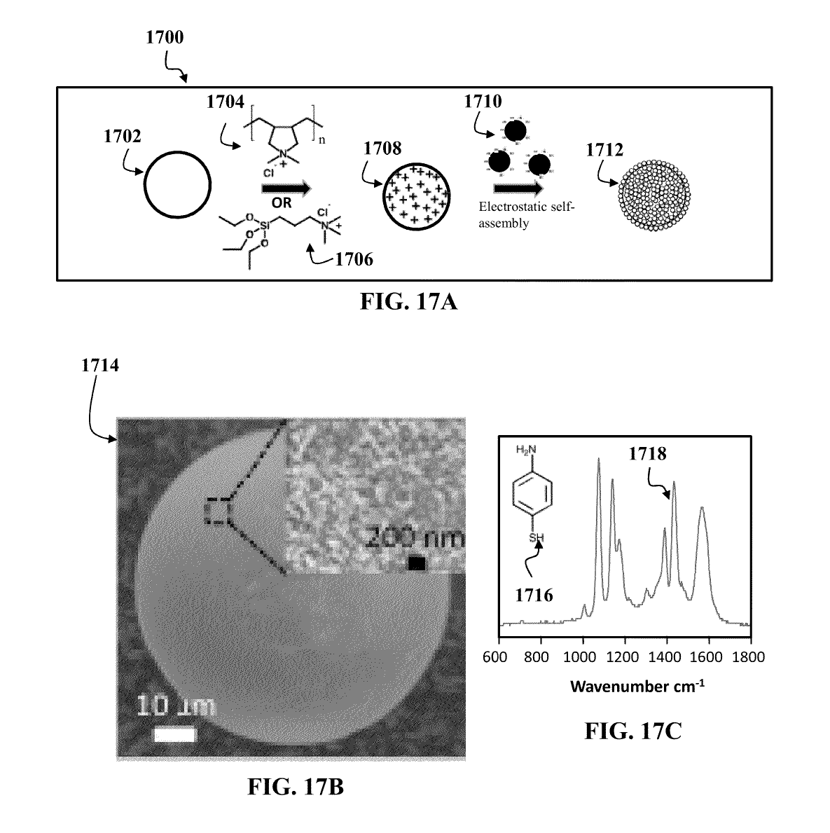

In one embodiment, a method is set forth for synthesizing SERS-active buoyant particles for use in detecting analytes as generally illustrated in FIGS. 2A-D and FIG. 3. Referring to FIG. 15, a method 1500 of preparing a SERS-active buoyant particle is shown. The method 1500 of FIG. 15 will be further described with reference to FIGS. 16A-C, which illustrates a general scheme for the synthesis of SERS-active buoyant particles, and FIGS. 17-19, which illustrate specific methods for synthesizing SERS-active buoyant particles. In step 1502 of method 1500, a buoyant particle is selected based on particle diameter, particle density, and chemical composition. Step 1502 takes into consideration the buoyancy of the particle in a specified liquid, such as water. In step 1504 of method 1500, the buoyant particle is then chemically modified to provide binding agents on the surface of the buoyant particle that is compatible with binding a SERS-active component, which is done in step 1506 of method 1500. Referring to FIG. 16A, buoyant particle 1600 is modified with binding agent 1602, which has a first chemical group 1604 that binds to the surface of buoyant particle 1600, and a second chemical group 1606 that binds to the surface of a SERS-active component 1608 to provide SERS-active buoyant particle 1610.

By way of illustration, FIG. 16B shows a set of representative scanning electron microscopy (SEM) images 1612 of SERS-active buoyant particles having a coating of gold nanoparticle. In this figure four SEM images are shown for three different SERS-active buoyant particle preparations using the general methods presented in FIGS. 15-16A-C, wherein the surface coverage of the gold nanoparticles (˜50 nm in diameter) on the buoyant particles increases as shown by 1614. FIG. 16C shows the effect that nanoparticle surface coverage has on the Raman spectral signature 1616 of a compound 1618 bound to the SERS-active buoyant particle 1610. That is, as surface coverage nanoparticle surface coverage increases, so does the intensity of the Raman signal 1616. This enhanced Raman signal is also related to the concepts presented in FIG. 3.

FIG. 17A outlines a method 1700 for preparing SERS-active buoyant particle 1712 using the general methods illustrated in FIGS. 15 and 16A. Method 1700 represents the synthesis of highly SERS-active buoyant particles prepared using a step-wise process starting with a buoyant particle 1702 which, in one embodiment, is a silicon dioxide bubble (available from 3M Corporation). The buoyant particle 1702 is chemically modified with a binding agent 1704 or 1706 to provide a positively charged intermediate buoyant particle 1708 having a highly cationically charged surface. Binding agent 1704 (e.g., polydimethyldiallylammonium chloride) binds electrostatically to the silicon dioxide, which initially has low negative charge density, to give a highly positive surface charge density. Similarly, addition of binding agent 1706 to the silicone dioxide bubble using standard silane coupling chemistry (Journal of the American Chemical Society, 2009, 131, 8746) provides the highly positive surface charge density shown in FIG. 17A (intermediate buoyant particle 1708). The resulting positively charged intermediate buoyant particle 1708 is then treated with a solution of negatively charged silver nanoparticles 1710 to give SERS-active buoyant particle composite 1712, which exhibits a conformal coating of silver nanoparticles as shown in SEM images 1714 in FIG. 17B. As shown in FIG. 17C, addition of the resulting SERS-active buoyant particle composites 1712 into a liquid sample containing a generic analyte 1716 at a concentration of 100 micromolar produces a strong Raman spectral signature 1718 after performing the general detection methods illustrated in FIGS. 1 and 2.

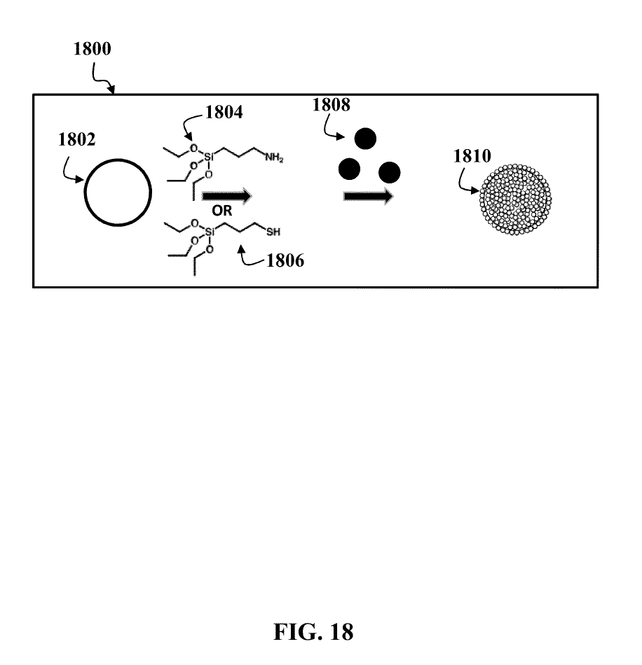

FIG. 18 outlines a method 1800 for preparing SERS-active buoyant particles using the general methods illustrated in FIGS. 15 and 16A. Method 1800 represents another synthesis of highly SERS-active buoyant particles prepared using a step-wise process starting with a buoyant particle 1802, which in one embodiment is a silicon dioxide bubble (available from 3M Corporation). The buoyant particle 1802 is chemically modified with a binding agent 1804 or 1806 using standard silane coupling chemistry (Journal of Materials Chemistry, 1997, 7, 259) to provide an intermediate buoyant particle having amine surface functional groups 1808 or thiol surface functional groups 1810, respectively. Amine groups are well known to bind gold particles, and thiol groups are well known to bind both gold and silver particles. The resulting intermediate buoyant particles are then treated with a solution of negatively charged gold or silver nanoparticles 1808 to give SERS-active buoyant particle composite 1810. In the method that uses chemical moiety 1804 and gold nanoparticles, the resulting SERS-active buoyant particles 1810 exhibit a conformal coating of gold nanoparticles, which is shown in the SEM images 1612 presented in FIG. 16B.

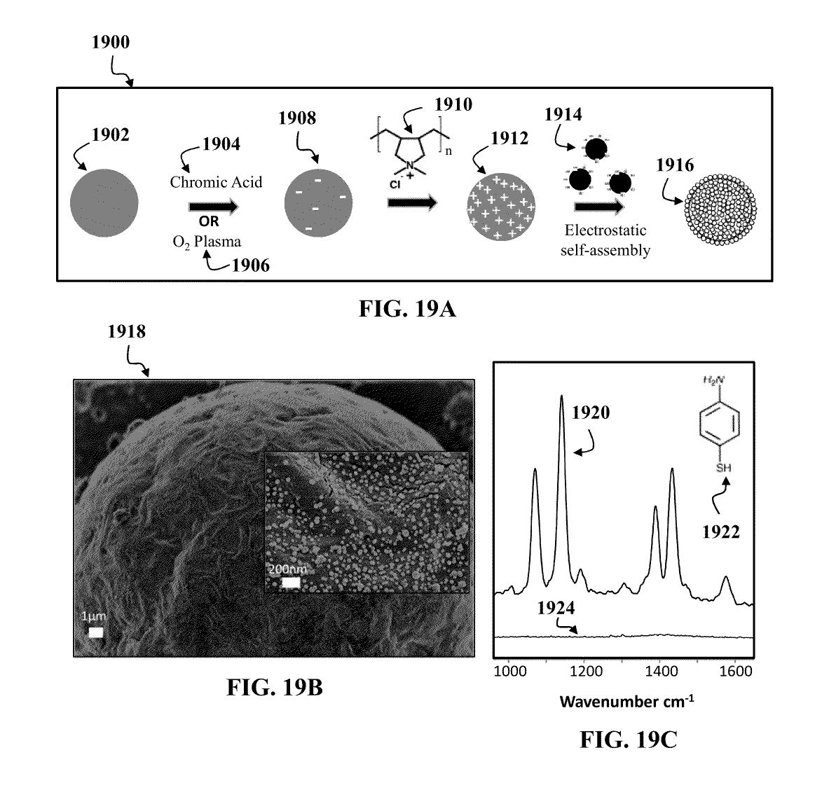

FIG. 19A outlines a method 1900 for preparing SERS-active buoyant particles using the general methods illustrated in FIGS. 15 and 16A. Method 1900 represents the synthesis of highly SERS-active buoyant particles prepared using a step-wise process starting with a polyethylene buoyant particle 1902 (available from Cospheric LLC). By way of example, the buoyant particle 1902 is chemically modified with either a solution phase chromic acid 1904 or oxygen plasma 1906 to provide a negatively charged intermediate buoyant particle 1908. Addition of a cationic polymer chemical moiety 1910 (e.g., polydimethyldiallylammonium chloride) provides a highly cationically charged intermediate buoyant particle 1912 as previously described in FIG. 17A (method 1700). The resulting positively charged intermediate 1912 is then treated with a solution of negatively charged nanoparticles 1914 to give a SERS-active buoyant particle 1916. FIG. 19B shows representative SEM images 1918 of SERS-active buoyant particles prepared according to method 1900 using 20-40 micrometer diameter polyethylene particles, chromic acid chemical modification and silver nanoparticles.

Presented in FIG. 19C are representative Raman spectra of a liquid sample of a generic analyte 1922 at a concentration of 100 micromolar in the presence of SERS-active buoyant particles 1916 (i.e., spectrum 1920), and in the presence of buoyant particle 1902 (i.e., spectrum 1922). These spectra illustrate the effect that SERS-active silver or gold coatings have on the Raman spectral signal intensity of bound analytes.

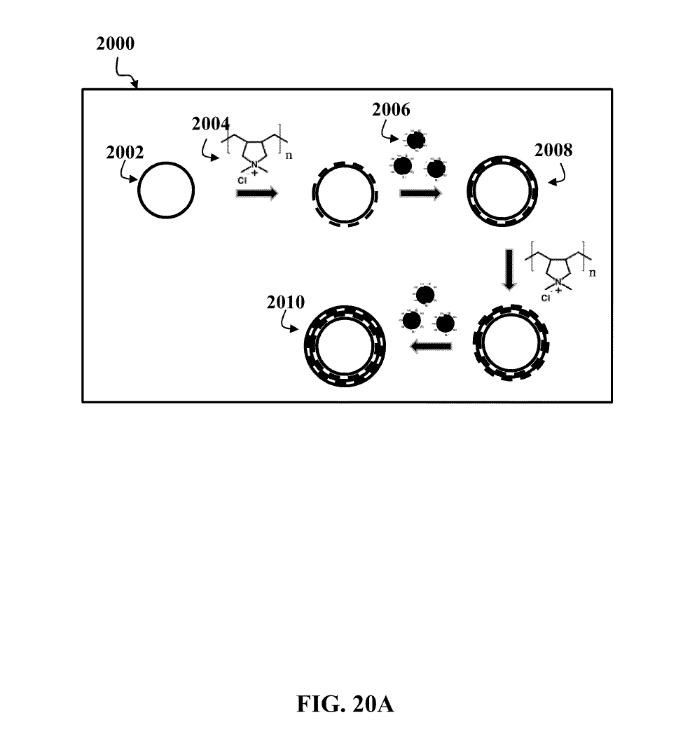

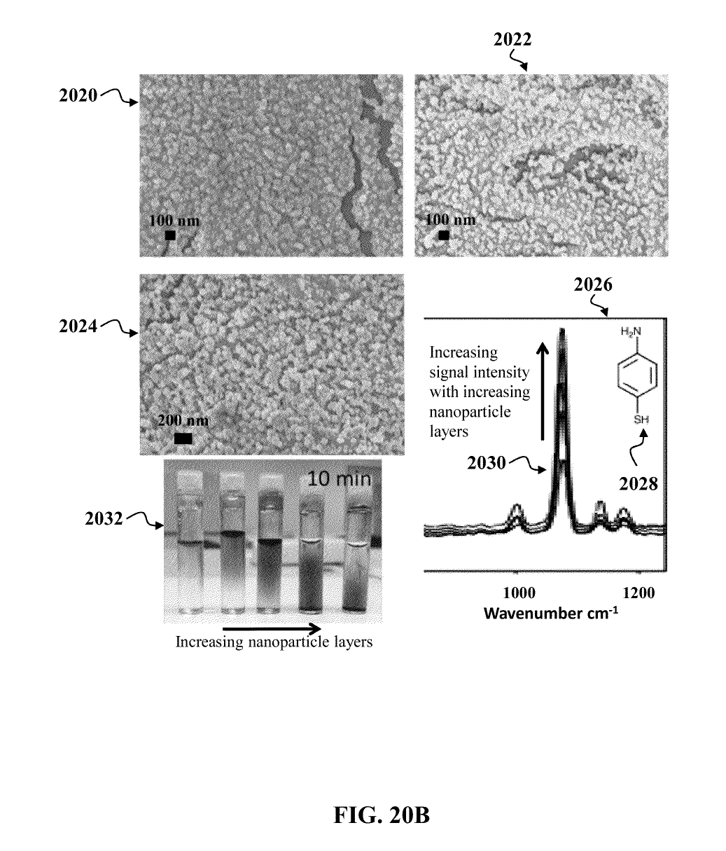

In one aspect of the invention, the specific SERS-active buoyant particles prepared using method 1900 and polyethylene-based buoyant particle 1902 can be further coated with silver or gold nanoparticles in order to produce SERS-active buoyant particles having multilayers of the nanoparticles. This process and the associated characteristics of these SERS-active buoyant particles are illustrated in FIG. 20A-B and discussed in greater detail in Example 4 of the specification. Of note is the affect that the multilayers have on the density of the SERS-active buoyant particles as shown in 2032 of FIG. 20B, wherein some of the particles are exhibiting neutral buoyancy.

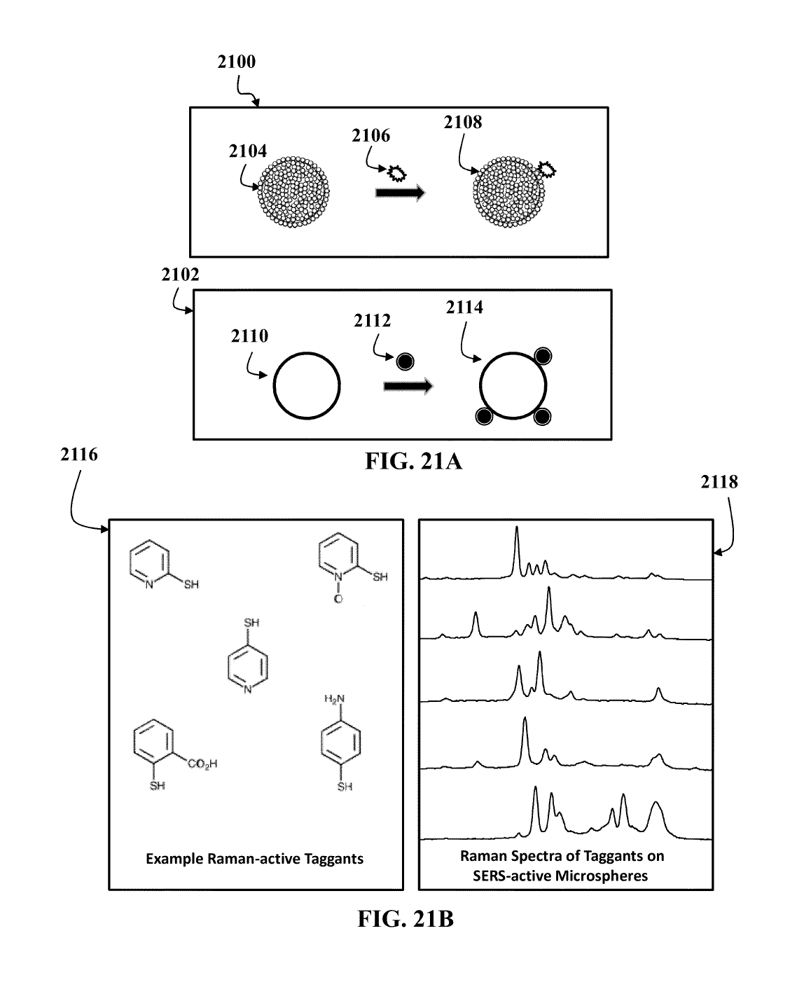

Referring to methods 2100 and 2102 (FIG. 21A), in another aspect of the invention, buoyant particles 2104 or 2110 can be modified with Raman-active components 2106 or 2112, respectively, each having a known Raman spectral signature. The resulting buoyant particle composites 2108 or 2114 are referred to as tagging buoyant particles. It is preferable that the tagging buoyant particles are neutrally buoyant and remain suspended in a liquid sample for a specified amount of time. In one aspect, buoyant particle 2104 is a multilayered SERS-active buoyant particle as described in FIG. 20 and the Raman-active component 2106 is a Raman-active molecule that is capable of binding gold or silver surfaces. In another aspect, the Raman-active component 2112 is SERS nanoparticle reporter as generally defined in FIG. 10 and is comprised of a gold or silver core material having a surface bound Raman-active molecule. Examples of Raman-active molecules that may be utilized in methods 2100 and 2102 are shown in 2116 and their corresponding Raman spectral signatures are shown in 2118. In one embodiment of the invention, tagging buoyant particles 2108 or 2114 can be used to tag a specified liquid sample to provide the liquid sample with a known Raman spectral signature.

### Example 1

### Preparation of Ag or Au-Coated SiO₂ Bubbles: SERS-Active Buoyant Particles Using Polymer-Based Chemical Modification and Electrostatic Self-Assembly of Charged Nanoparticles

The following example describes a method for depositing gold and silver nanoparticle coatings onto the surface of SiO₂ hollow microspheres—referred to herein as glass bubbles (commercially available from 3M Corporation). The process used is outlined generally in FIG. 16A and in more specifically in FIG. 17A. Briefly, 0.5 grams of glass bubble was combined with 50 mL of a 1% by weight aqueous solution of polydiallyldimethylammonium chloride (PDADMAC). In this step, cationic PDADMAC is charge coupling with the ill-defined negative charge on the surface of the SiO₂ bubbles to provide a surface with a high positive charge density as illustrated in FIG. 17A for intermediate buoyant particle 1708. This mixture was agitated on a bench top shaker for 30 minutes and allowed to rest for 30 minutes to allow bubbles to float to the top of the solution. After this time, the bottom aqueous layer was drawn off using a syringe and the remaining floating bubbles were combined with 50 mL of distilled water, agitated for approximately 1 minute, allowed to rest, and the water layer was removed as before. This washing process was repeated two additional times to remove excess PDADMAC.

At this point, a volume of hydrated bubbles was then taken on to nanoparticle deposition. The amount of hydrated bubbles used for nanoparticle deposition was estimated by removing a volume and drying them to a constant weight. In one example, 25 mg of S60HS 3M bubbles were combined with 50 mL of an aqueous suspension of gold nanoparticle having a average diameter of 50 nm. Negatively charged, aqueous gold nanoparticle was prepared via citrate reduction according to a published procedure (Nature, 1973, 241, 20-22). The resulting mixture was agitated on a benchtop shaker until bubbles were saturated with nanoparticles. Excess nanoparticle was removed by washing the resulting SERS-active buoyant particles with water has described above for PDADMAC. Scanning electron microscopy analysis (FIG. 16B) indicated a bubble surface highly decorated with gold nanoparticles. In another example, silver nanoparticle-coated K25 3M bubbles were prepared using the method described above for gold. Approximately 45 nm diameter, negatively charged aqueous silver nanoparticle was prepared via citrate reduction using a published procedure (Journal of Physical Chemistry 1982, 86, (17), 3391-3395). Representative SEM images of the resulting silver SERS-active buoyant particles are presented in FIG. 17B.

### Example 2

### Preparation of Ag or Au-Coated SiO₂ Bubbles: SERS-Active Buoyant Particles Using Silane-Based Chemical Modification of SiO₂ and Nanoparticle Deposition

The following example describes another method for preparing SERS-active buoyant particles. This method is similar to example 1, but utilizes silane chemistry to chemically modify the surface of SiO₂ bubbles. Briefly, 0.5 grams of SiO₂ was incubated overnight at room temperature in 50 mL of 2.5 M NaOH with shaking. The resulting bubbles were allowed to rest to facilitate bubble separation, and the floating bubbles were washed with water as described above in example 1. The hydrated bubbles were then immersed in 0.1M aqueous HCl for 10 minutes. After this time, the aqueous layer was removed for the floating bubbles, followed by washing the bubbles in water two times, and then washed in methanol two additional time. Bubbles were stored in methanol until further use. Bubbles were then silanized with either (3-aminopropyl)triethoxysilane (refer to 1804 shown in FIG. 18) or with N-Trimethoxysilyl-N,N,N-trimethylammonium chloride (refer to 1706 in FIG. 17A). For the silanization step, bubbles were combined with 50 mL of a 1% aqueous solution of the appropriate silane and agitated at room temperature by shaking for 15 minutes. After this time, excess aqueous silane was removed by syringe, and the hydrated bubbles were baked at 110-120° C. to facilitate silane binding to the bubble surface. Chemically modified bubbles were then combined with negatively charged aqueous gold or silver nanoparticle suspensions to give gold or silver coated SERS-active buoyant particles as described in example 1. When using 1804 as the linker molecule the nanoparticle depositions works though a ligation process wherein the amine groups of 1804 bind strongly to the gold nanoparticles. When using 1706 as the linker molecule, the silver and gold deposition occurs via electrostatic self-assembly between positively charged bubbles and negatively charged gold or silver nanoparticles.

### Example 3

### Preparation of Ag or Au-Coated Polyethylene Microspheres SERS-Active Buoyant Particles

The following example describes the preparation of SERS-active buoyant particles using polyethylene microspheres using different types of chemical modifications of polyethylene followed by Au or Ag nanoparticle deposition. Polyethylene is a fairly inert material and requires chemical modification to allow for Au/Ag binding. Several methods are available for chemically modifying polyethylene microspheres and described herein. In one method, negatively charged polyethylene microsphere were prepared by adding 100 mg of polyethylene microspheres (available from Cospheric LLC) were added to a chromic acid solution comprised of 0.5 mL water, 0.25 g chromium trioxide, and 0.136 mL of sulfuric acid. The mixture was heated on a hot plate at ˜150° C. for 2 hours with stirring. The resulting oxidized polyethylene microspheres were then collected by filtration and washed with copious amounts of water to furnish negatively charged polyethylene, which was subsequently combined with aqueous 1% PDADMAC to render the microspheres cationic as described above in example 1 for SiO₂ bubbles.

In another method, negatively charged polyethylene microsphere were prepared using oxygen plasma. Briefly, 50 mg of polyethylene microspheres were weighed and spread out evenly on the surface of a microscope glass slide and placed inside a Harrick oxygen plasma cleaner oven and treated for 1 min at HIGH power and 500 mtorr to give negatively charged polyethylene microspheres. After plasma treatment, the microspheres were immersed in 1% PDADMAC solution (50 mL) and agitated on shaker 30 min. as described previously. Gold or silver-coated polyethylene microspheres were prepared as previously described in examples 1 and 2. Given that their density is ˜1 g/cc, the various coating and washing steps were performed by collecting the microspheres via filtration at each step in the process. FIG. 19B shows representative SEM images of the resulting SERS-active buoyant particles.

### Example 4

### Preparation of SERS-Active Buoyant Particles Having Au or Ag Nanoparticle Multilayers Using Layer-by-Layer Self-Assembly

The SERS-active buoyant particles described in examples 1-3 can be further modified with gold or silver nanoparticles using additional coating steps of PDADMAC and negatively charged nanoparticles. In each example 1-3, the resulting SERS-active buoyant particle has a net negative surface charged due to the presence of citrate on the surface of the gold or silver particles. Thus, these negatively charged SERS-active buoyant particles can be iteratively treated with 1% PDADMAC and negatively charged Au or Ag nanoparticles to give SERS-active buoyant particles having multiple layers of gold or silver nanoparticles as shown in FIG. 20A. In this example, SERS-active buoyant particle 2002 having a single layer of silver nanoparticles was used as starting material. For example, 10 mg of the SERS-active buoyant particles described in example 3 were immersed in 1 mL of 1% PDADMAC 2004 and agitated for 30 min. After this time the buoyant particles were washed with copious amounts of water and treated with 10 mL of negatively aqueous silver nanoparticle 2006 to give SERS-active buoyant particle 2008 having two layers of silver nanoparticle. This process was repeated again to give a third layer of silver nanoparticles as shown on SERS-active buoyant particle 2010.

In this way, alternating layers of PDADMAC and silver nanoparticle were done until 5 layers of silver nanoparticles were coated onto the buoyant particle. Representative SEM images for 2 (2020), 3 (2022), and 4 (2024) layers of nanoparticles are shown in FIG. 20B. Also shown in FIG. 20B representative Raman analysis (2026) of the various SERS-active buoyant particles having 1-5 layers of silver nanoparticles. Raman spectra (2030) of the various layered SERS-active buoyant particles in the presence of a model analyte 2028 for 1, 2, 3, 4, and 5 layers of nanoparticles are presented in FIG. 20B.

The production of SERS-active buoyant particles having multilayers of silver or gold nanoparticles as described above and depicted in FIG. 20 can also be used to affect the density of the resulting SERS-active buoyant particle. The sample tubes pictured in FIG. 20B (referring to 2032) show how the multilayers affect particle density, and thus, the propensity of the SERS-active buoyant particles to remain suspended in a liquid sample (in this case, the picture was captured 10 minutes after shaking the particles and letting them rest). This neutral buoyancy property exhibited by a few of the samples picture in 2032 is a desirable feature for various tagging applications as described previously in this specification and illustrated in FIGS. 21A-B.

### Example 5

### Detection of Cyanide Using Gold-Coated SERS-Active Buoyant Particles

The following example describes the use of SERS-active buoyant particles for detecting cyanide in liquid samples, herein referred to as a cyanide LoB-MR detection system (illustrated in FIG. 5). In this assay, cyanide is detected based on its inherent binding affinity for gold (cyanide is used in gold mining given its ability to bind and dissolve gold), and the intrinsic Raman signal associated with TN. Briefly, SERS-active buoyant particles prepared according to example 2 and were comprised of S60HS glass bubbles (3M Corp.) having a conformal coating of 50 nm gold nanoparticles. The SERS-active buoyant particles were combined with aqueous solutions of sodium cyanide, which produced the diagnostic Raman band for CN at 2125 cm⁻¹ (refer to the Raman spectrum in FIG. 5). A calibration curve and limit of detection (LOD) for cyanide was established by combining a set number of SERS-active buoyant particles with known concentrations of cyanide, and the signal intensity of the 2125 cm⁻¹ peak was plotted as a function of cyanide concentration (refer to plot in FIG. 5). The resulting data shows a linear signal response between 0-10 ppb ⁻CN as well as a dramatic drop in signal beyond ˜10 ppb—this drop in signal is likely a result of loss of gold due to dissolution (i.e., CN⁻ is dissolving gold particles at the higher concentrations). Using the linear region of this plot, a LOD was calculated using the following equation:

LOD=3σ/m

where σ is the standard deviation in the y-intercept, the number 3 corresponds to a confidence interval of 99.5% and m is the slope. For example, in an assay using ˜1000 LoBs in a sample volume of 100 μL, a detection limit of 173 parts-per-trillion was calculated for cyanide.

### Example 6

### Detection of Cortisol Using a Competitive Immunoassay: a LoB-MR Detection System Using Silver-Coated SERS-Active Buoyant Particles and a Competing Raman-Active Cortisol Analyte

Cortisol is a key glucocorticoid hormone that plays a role in important metabolic (e.g., gluconeogenesis) and immunologic (e.g., immunosuppression and disease resistance) actions. Cortisol levels in human plasma are elevated in response to stress, and though this is an important physiological process (e.g., cortisol aids in protein/energy production under stressful conditions), chronically high levels of cortisol can lead to increased risks of developing numerous heath problems, including: obesity, heart disease, digestive problems, memory impairment, and Cushing's syndrome. Cortisol levels have also been linked to post-traumatic stress disorder (PTSD). There is a need in developing methods for detecting low and high levels of blood cortisol.

A LoB-MR competitive immunoassay is described below and illustrated in FIG. 8, which is comprised of SERS-active glass bubbles (specifically, 3M S60H bubbles coated with 45 nm gold nanoparticles using the method described in example 1), an anti-cortisol antibody (808) bound to the SERS-active glass bubbles, and a competing Raman-active cortisol reporter (800). Ag-coated SERS-active glass bubbles were functionalized with a monoclonal primary antibody (mAb) against cortisol available from US Biologicals (raised in mouse, C7904-11B). Briefly, SERS-active glass bubbles were reacted with a 0.3% by weight solution of the sodium salt of polyacrylic acid (PAA; average molecular weight 450,000; SigmaAldrich) for 30 minutes. After this time the SERS-active bubbles were washed copious amounts of water to remove excess PAA, and subsequently coupled with the anti-cortisol antibody using 1-Ethyl-3-[3-dimethylaminopropyl]carbodiimide and N-hydroxysulfosuccinimide (EDC-NHS) coupling chemistry.

A competing cortisol reporter (800) was prepared starting from the 3-(O-carboxymethyl)oxime derivative of cortisol (Sigma-Aldrich; St. Louis, Mo.). Using standard coupling chemistry, the oxime derivative was converted to the competing cortisol reporter via EDC/NHS coupling with a commercially available Fluorescein cadaverine (Invitrogen, Inc.), which exhibits a known Raman spectrum. To demonstrate the competitive immunoassay, the 1 mg of anti-cortisol antibody coated SERS-active bubbles described above were combined with 50 microliters of competing cortisol reporter 800 at a concentration of 100 micromolar, and portion of the resulting SERS-active buoyant particle composite 810 was drawn up into a microcapillary tube. The buoyant particles were then allowed to localize and concentrate to the top of the tube and subsequently analyzed using a handheld Raman spectrometer (DeltaNu ReporteR; 785 nm excitation). FIG. 8 shows the resulting spectrum (816) for this composite. Next, excess solution from the capillary tube containing the 810 composite was removed and the composite was washed with water to remove excess 800. A solution of cortisol at a concentration of 100 micromolar was then drawn up in the same capillary and mixed. The bubbles were again allowed to localize and concentrate to the top of the tube and subsequently analyzed by Raman. FIG. 8 shows the resulting spectrum (814) from this analysis. A comparison of the spectra plotted in FIG. 8 indicates that the signal intensity is decreasing, which is presumably due to displacement of 800 from the antibody due to free cortisol 806 to form buoyant composite 812.

### Example 7

### Detection of Cholera Toxin Using a Sandwich Immunoassay: a LoB-NPR Detection System Using SiO₂ Buoyant Particles and a SERS Nanoparticle Reporter

Vibrio cholera is a gram-negative bacterium that can cause the disease cholera, a highly contagious and commonly fatal infection of the gastrointestinal tract. The cholera toxin (CT) appears to be necessary to cause disease in humans, and is found in various infectious strains. V. cholerae is commonly found in salt water, estuarial water, fresh water and even ground water and there is a need for the development of methods for detecting this causative agent. The following example describes a sandwich immunoassay method for detecting CT using the LoB-NPR detection system illustrated in FIGS. 11 and 12. The LoB-NPR assay reagents were prepared using standard coupling chemistries. Briefly, S60HS silica bubbles (3M Corp.) were activated with (3-aminopropyl)triethoxysilane (refer to 1804 shown in FIG. 18), and subsequently coupled with cholera toxin subunit B antibody (anti CT antibody) using standard EDC-NHS chemistry. Nanoparticle reporters were prepared by first synthesizing 60-75 nm AuNPs, applying a coating of bispyridylethylene (BPE; a well-known Raman-active reporter molecule), and formation of a stabilizing SiO₂ shell on the surface of the AuNP. Nanoparticle reporters were then functionalized with anti-CT antibody using simple physisorption to give CT-specific nanoparticle reporters.

When anti-CT coupled glass bubbles are combined with an aqueous sample containing anti-CT coupled nanoparticle reporters and CT antigen, buoyant particle composite 1202 is formed as shown in FIG. 12A. Formation of this composite produces a spectrum typical of that shown in FIG. 12A (e.g., 1208). Also shown in this figure are SEM images (1206) of the resultant buoyant particle composite.

Using the method of standard additions, a calibration plot (1210) for CT antigen was produced by starting from an initial sample containing known concentrations anti-CT modified bubbles, CT antigen and anti-CT modified nanoparticle reporters. Additional aliquots of CT antigen were then added and the resulting Raman signal corresponding to formation of more buoyant particle composite 1202 was plotted. The value of CT antigen contained in the initial solution was then back calculated by extrapolating the line to the x-intercept or by the equation [c]=b/m, where [c] is the unknown concentration, b is the y-intercept, and m is the slope (refer to 1210 in FIG. 12D). The y-axis in this analysis is the ratio of the SiO₂ glass peak around 1000 cm⁻¹ and our reporter molecule, BPE, peak around 1600 cm⁻¹. Using this methodology and a linear regression, the initial CT antigen was calculated to be 3700 ng (actual 5000 ng). Using this plot and a standard equation (3×σ/m; described in example 5), the limit of detection (LOD) for CT antigen was calculated to be 1100 ng.

While the invention has been described with reference to certain embodiments, it will be understood by those skilled in the art that various changes may be made and equivalents may be substituted for elements thereof to adapt to particular situations without departing from the scope of the disclosure. Therefore, it is intended that the claims not be limited to the particular embodiments disclosed, but that the claims will include all embodiments falling within the scope and spirit of the appended claims.

What is claimed is:

1. A method for detecting at least one analyte of interest in a sample, the method comprising:
contacting a liquid sample having a first density with at least one buoyant particle with a second density and a first chemical moiety disposed on a surface of the buoyant particle, the first chemical moiety being chosen to selectively bind to an analyte, the second density being less than the first density;
permitting the first chemical moiety to bind to the analyte in the liquid sample to form a buoyant particle composite;
determining a discrete location in the liquid sample where the buoyant particle composite will be localized based on the first density of the liquid sample and a density of the buoyant particle composite, respectively;
detecting the analyte in the liquid sample by interrogating the discrete location with a Raman spectrometer.

2. The method of claim 1, wherein the liquid sample is at least 90% water by volume.

3. The method of claim 1, wherein the liquid sample is at least 10% hydrocarbon solvent by volume.

4. The method of claim 1, wherein the buoyant particle has a cross-sectional diameter within the range of 0.1 micrometers and 500 micrometers.

5. The method of claim 1, wherein the second density of the buoyant particle is within the range of 0.1 and 1.5 g per mL.

6. The method of claim 1, wherein the buoyant particle is comprised of silica dioxide, aluminum oxide, titanium dioxide, silver, gold, or combinations of the same.

7. The method of claim 1, wherein the buoyant particle is comprised of an organic polymer.

8. The method of claim 1, wherein the buoyant particle is a gas filled hollow particle.

9. The method as recited in claim 1, further comprising the step of coating the at least one buoyant particle with a SERS-active coating prior to the step of contacting the liquid sample with the at least one buoyant particle, the SERS-active coating comprising particles selected from the group consisting of gold particles, silver particles, and combinations thereof.

10. The method as recited in claim 9, wherein the first chemical moiety is the surface of the SERS-active coating.

11. The method as recited in claim 10, wherein the step of permitting the first chemical moiety to bind to the analyte directly binds the analyte to the SERS-active coating, the analyte being Raman-active with a measurable Raman spectral signature.

12. The method as recited in claim 1, the method further comprising the steps of
contacting the liquid sample with at least one Raman-active component having a second chemical moiety that binds to the analyte, the Raman-active component having a Raman spectral signature; and
permitting the second chemical moiety to bind to the analyte in the liquid sample, thereby tethering the Raman-active component to the buoyant particle to form the buoyant particle composite.

13. The method as recited in claim 12, wherein the Raman-active component is a SERS nanoparticle reporter comprised of a gold or silver core, the Raman-active component having at least one Raman-active compound that is surface-bound to the gold or silver core.

14. The method as recited in claim 12, further comprising the step of coating the at least one buoyant particle with a SERS-active coating prior to the step of contacting the liquid sample with the at least one buoyant particle, the SERS-active coating comprising particles selected from the group consisting of gold particles, silver particles, and combinations thereof.

15. The method as recited in claim 12, wherein the Raman-active component is comprised of at least one Raman-active molecule with a Raman spectral signature.

16. The method as recited in claim 12, wherein the analyte is an antigen and the first chemical moiety and the second chemical moiety comprise antibodies chosen to selectively bind to the analyte.

17. The method as recited in claim 12, wherein the analyte is an antibody and the first chemical moiety and the second chemical moiety comprise antigens chosen to selectively bind to the analyte.

18. The method as recited in claim 12, wherein the analyte includes a first nucleotide and the first chemical moiety and the second chemical moiety comprise one or more second nucleotides chosen to selectively bind the analyte.

19. The method of claim 1, wherein the analyte is detected indirectly through a competitive process of forming two different buoyant particle composites, the method including the steps of:
contacting the liquid sample with a Raman-active component having a second chemical moiety comprised of a competing analyte, the competing analyte chosen to competitively bind with both the first chemical moiety and the second chemical moiety;
permitting competitive binding to occur between the analyte, the competitive analyte and the first chemical moiety and the second chemical moiety;
determining the ratio of analyte to competing analyte that are bound to the buoyant particle.

20. The method of claim 19, wherein the analyte is an antigen and the first chemical moiety and the second chemical moiety comprise antibodies.

21. The method of claim 19, further comprising the step of coating the at least one buoyant particle with a SERS-active coating prior to the step of contacting the liquid sample with the at least one buoyant particle, the SERS-active coating comprising particles selected from the group consisting of gold particles, silver particles, and combinations thereof, the Raman-active competing analyte comprises at least one Raman-active molecule with a Raman-active spectral signature.

22. The method of claim 19, wherein the Raman-active competing analyte comprises at least one SERS nanoparticle reporter comprised of a gold or silver core having at least one surface bound Raman-active compound with a Raman spectral signature.

23. A method of producing a SERS-active buoyant particle comprising the steps of:
specifying a liquid;
providing a buoyant core material that is buoyant in the specified liquid;
immobilizing a gold or silver SERS-active coating onto the core material to provide a SERS-active particle; and
bonding at least one chemical moiety to the SERS-active particle, wherein the chemical moiety can specifically bind to at least one analyte of interest.

24. The method as recited in claim 23, further comprising the step of binding at least one Raman-active component to the SERS-active coating to give a SERS-active buoyant particle having a known Raman spectral signature.

25. The method as recited in claim 24, wherein the SERS-active buoyant particle having a known Raman spectral signature is neutrally buoyant in a specified liquid.

26. A hollow, gas filled surface-enhanced Raman scattering (SERS)-active composite particle, comprising:
a core material encapsulating at least one type of gas of a specified density; and
a SERS-active coating comprised of gold or silver immobilized onto the core material.
